SEQUENCE LISTING (23 sequences)

SEQ ID NO: 1 - protein, 745 aa, Komagataeibacter xylinus
MSEVQSSAPAESWFGRFSNKILSLRGASYVVGALGLCALLAATMVTLSLNEQMIVALVCVAVFFIVGRRKSRRTQVFLEVLSALVSLRYLTWRLTETLDFDTWTQGILGVTLLLAELYALYMLFLSYFQTISPLHRAPLPLPANPDEWPTVDIFIPTYDEALSIVRLTVLGALGIDWPPDKVNVYILDDGRREEFARFAEACGARYIARPDNAHAKAGNLNYAIKHTTGDHILILDCDHIPTRAFLQISMGWMVSDSNIALLQTPHHFYSPDPFQRNLAVGYRTPPEGNLFYGVIQDGNDFWDATFFCGSCAILRRKAIEEIGGFATETVTEDAHTALRMQRKGWSTAYLRIPLASGLATERLITHIGQRMRWARGMIQIFRVDNPMLGSGLKLGQRLCYLSAMTSFFFAIPRVIFLASPLAFLFFSQNIIAASPLAVGVYAIPHMFHSIATAAKVNKGWRYSFWSEVYETVMALFLVRVTIVTMLFPSKGKFNVTEKGGVLEREEFDLTATYPNIIFAIIMALGLLRGLYALIFQHLDIISERAYALNCIWSVISLIILMAAISVGRETKQLRQSHRIEAQIPVTVYDYDGNSSHGITEDVSMGGVAIHLPWREVTPDHPVQVVIHAVLDGEEMNLPATMIRSAQGKAVFTWSISNIQVEAAVVRFVFGRADAWLQWNNYEDDRPLRSLWSLILSIKALFRRKGQMIAHSRPKKKPIALPVERREPTTSQGGQKQEGKISRAAS

SEQ ID NO: 2 - DNA, 2238 bp, Komagataeibacter xylinus
atgtcagaggttcagtcgtcagcgcctgcggaaagctggttcggccgcttttccaacaagatactgtcactgcgcggtgccagctatgtcgttggggcgttggggctttgcgccctgcttgccgcaaccatggttacgctgtcgcttaatgaacagatgattgtggcattagtgtgtgtggcggtgttttttatcgtcggccgccgcaaaagccgtcgcacccaggtctttctggaggtgctgtcggcgctggtgtccctgcggtatctgacgtggcggctgacggaaacgctggactttgatacctggacgcagggcatcctgggtgtcacgctgctgctggcggaactgtatgcgctctacatgctgttcctcagttatttccagacgatttcccccctgcatcgtgcgccgctgccgctgccggccaatcccgatgagtggcccacggttgatattttcatcccgacctatgacgaagcactgagcatcgtgcgtctgacggtgctcggagcgttgggtatcgactggccgcctgataaggtgaacgtctatattctggatgacggcaggcgtgaggaattcgcccgttttgccgaggcctgcggcgcgcgttacatcgcccgtcccgataacgcgcacgccaaggccggtaacctgaactacgccattaaacataccacgggcgatcacatcctcatcctggactgtgaccatatcccgacgcgtgctttcctgcagatctccatgggctggatggttagcgattcgaacatcgccctgctgcagacgccgcatcacttctattcccccgatccgttccagcgtaacctggcggtggggtaccgcaccccgcccgaaggcaatctgttctatggcgtcattcaggacggtaacgacttctgggatgcgaccttcttctgcggatcgtgcgccatcctgcgccgtaaggcgattgaggaaatcggcggcttcgcaaccgaaaccgtgacagaagacgcccataccgcgctgcgtatgcagcgcaagggctggtcgaccgcctacctgcgcattccgctggccagtggtctggcgacagaacgtctcattacgcatatcgggcagcgtatgcgctgggcccgtggcatgatccagattttccgcgttgataacccgatgcttggctcgggtctgaagcttgggcagcgtctttgctacctgtcggccatgacgtcgttcttcttcgccattccgcgcgtcatcttccttgcatccccgctggccttcctgttcttcagccagaatatcatcgcggcatccccgctggcggtgggggtctacgccatcccgcacatgttccattccattgcgactgcggcgaaggtcaacaagggctggcggtattcgttctggagtgaagtgtacgaaaccgtcatggcgctgttcctggtgcgggtgaccatcgtcacgatgctgttcccctccaagggcaagttcaacgtgacggaaaaaggtggcgttctggaacgtgaggaattcgacctcaccgccacctatccgaatattattttcgccatcatcatggccctcggcctgttgcgtgggctatatgcgctgatcttccagcacctggacatcatttcggaacgtgcgtacgcgctgaactgcatctggtcggtgatcagtctgatcatcctgatggcggcaatctccgtggggcgtgaaacaaagcagctgcgccagagccatcgtatcgaagcccagatccccgttacggtttatgattacgatggcaattcgagccacggcattaccgaagacgtctccatgggcggtgtggcgatccacctgccatggcgtgaggttactcccgatcaccctgtacaggtcgtgatccatgccgtactggatggcgaggagatgaaccttccggccaccatgatccgcagtgcccagggcaaggcggtgtttacgtggtcgatcagtaacattcaggttgaggcggccgtggtccggtttgtgttcggacgcgccgatgcctggctgcagtggaataattatgaggatgaccggccgttacgaagcctgtggagtctgatcctcagcatcaaggcactgttccgcaggaagggtcagatgattgcccatagtcgtcccaaaaagaaaccaattgcactgccggttgagcgtagggagccaacaaccagccagggtggtcagaaacaggaaggaaagatcagtcgtgcggcctcgtga

SEQ ID NO: 3 - DNA, 39 bp, Artificial Sequence
  Synthetic gapA promoter F-primer
ctgccccccgagaccaacttcggcggcgcccgagcgtga

SEQ ID NO: 4 - DNA, 2238 bp, Artificial Sequence
  Synthetic Komagataeibacter xylinus modified
atgtcagaggttcagtcgtcagcgcctgcggaaagctggttcggccgcttttccaacaagatactgtcactgcgcggtgccagctatgtcgttggggcgttggggctttgcgccctgcttgccgcaaccatggttacgctgtcgcttaatgaacagatgattgtggcattagtgtgtgtggcggtgttttttatcgtcggccgccgcaaaagccgtcgcacccaggtctttctggaggtgctgtcggcgctggtgtccctgcggtatctgacgtggcggctgacggaaacgctggactttgatacctggacgcagggcatcctgggtgtcacgttgttactggcggaactgtatgcgctctacatgctgttcctcagttatttccagacgatttcccccctgcatcgtgcgccgctgccgctgccggccaatcccgatgagtggcccacggttgatattttcatcccgacctatgacgaagcactgagcatcgtgcgtctgacggtgctcggagcgttgggtatcgactggccgcctgataaggtgaacgtctatattctggatgacggcaggcgtgaggaattcgcccgttttgccgaggcctgcggcgcgcgttacatcgcccgtcccgataacgcgcacgccaaggccggtaacctgaactacgccattaaacataccacgggcgatcacatcctcatcctggactgtgaccatatcccgacgcgtgctttcctgcagatctccatgggctggatggttagcgattcgaacatcgccctgctgcagacgccgcatcacttctattcccccgatccgttccagcgtaacctggcggtggggtaccgcaccccgcccgaaggcaatctgttctatggcgtcattcaggacggtaacgacttctgggatgcgaccttcttctgcggatcgtgcgccatcctgcgccgtaaggcgattgaggaaatcggcggcttcgcaaccgaaaccgtgacagaagacgcccataccgcgctgcgtatgcagcgcaagggctggtcgaccgcctacctgcgcattccgctggccagtggtctggcgacagaacgtctcattacgcatatcgggcagcgtatgcgctgggcccgtggcatgatccagattttccgcgttgataacccgatgcttggctcgggtctgaagcttgggcagcgtctttgctacctgtcggccatgacgtcgttcttcttcgccattccgcgcgtcatcttccttgcatccccgctggccttcctgttcttcagccagaatatcatcgcggcatccccgctggcggtgggggtctacgccatcccgcacatgttccattccattgcgactgcggcgaaggtcaacaagggctggcggtattcgttctggagtgaagtgtacgaaaccgtcatggcgctgttcctggtgcgggtgaccatcgtcacgatgctgttcccctccaagggcaagttcaacgtgacggaaaaaggtggcgttctggaacgtgaggaattcgacctcaccgccacctatccgaatattattttcgccatcatcatggccctcggcctgttgcgtgggctatatgcgctgatcttccagcacctggacatcatttcggaacgtgcgtacgcgctgaactgcatctggtcggtgatcagtctgatcatcctgatggcggcaatctccgtggggcgtgaaacaaagcagctgcgccagagccatcgtatcgaagcccagatccccgttacggtttatgattacgatggcaattcgagccacggcattaccgaagacgtctccatgggcggtgtggcgatccacctgccatggcgtgaggttactcccgatcaccctgtacaggtcgtgatccatgccgtactggatggcgaggagatgaaccttccggccaccatgatccgcagtgcccagggcaaggcggtgtttacgtggtcgatcagtaacattcaggttgaggcggccgtggtccggtttgtgttcggacgcgccgatgcctggctgcagtggaataattatgaggatgaccggccgttacgaagcctgtggagtctgatcctcagcatcaaggcactgttccgcaggaagggtcagatgattgcccatagtcgtcccaaaaagaaaccaattgcactgccggttgagcgtagggagccaacaaccagccagggtggtcagaaacaggaaggaaagatcagtcgtgcggcctcgtga

SEQ ID NO: 5 - DNA, 33 bp, Artificial Sequence
  Synthetic gapA promoter R-primer
aaggtctgacatttcacacactgacatcggccg

SEQ ID NO: 6 - DNA, 2238 bp, Artificial Sequence
  Synthetic Escherichia coli codon optimized Komagataeibacter xylinus
atgagcgaggttcaaagcagcgcgccggcggaaagctggttcggtcgttttagcaacaagattctgagcctgcgtggtgcgagctacgtggttggtgcgctgggcctgtgcgcgctgctggcggcgacgatggtgaccctgagcctgaacgagcagatgatcgtggcgctggtgtgcgttgcggtgttctttattgttggtcgtcgtaaaagccgtcgtacccaggtgttcctggaagtgctgagcgcgctggtgagcctgcgttacctgacctggcgtctgaccgaaaccctggacttcgatacctggacccagggtattctgggcgttaccctgctgctggcggaactgtacgcgctgtatatgctgtttctgagctatttccaaaccatcagcccgctgcaccgtgcgccgctgccgctgccggcgaacccggatgagtggccgaccgtggacatcttcattccgacctacgacgaagcgctgagcatcgttcgtctgaccgttctgggtgcgctgggcattgattggccgccagacaaggttaacgtgtatatcctggacgatggccgtcgtgaggaatttgcgcgttttgcggaggcgtgcggtgcgcgttacattgcgcgtccggataacgcgcacgcgaaagcgggcaacctgaactatgcgatcaagcacaccaccggtgaccacatcctgattctggactgcgatcacatcccgacccgtgcgttcctgcagattagcatgggttggatggttagcgatagcaacatcgcgctgctgcagaccccgcaccacttttacagcccggacccgttccaacgtaacctggcggttggctaccgtaccccgccggagggtaacctgttttatggcgtgattcaggacggtaacgatttctgggacgcgaccttcttttgcggtagctgcgcgatcctgcgtcgtaaagcgatcgaggaaattggtggctttgcgaccgaaaccgtgaccgaagatgcgcacaccgcgctgcgtatgcagcgtaagggctggagcaccgcgtatctgcgtatcccgctggcgagcggtctggcgaccgaacgtctgatcacccacattggccagcgtatgcgttgggcgcgtggtatgatccaaattttccgtgttgacaacccgatgctgggtagcggcctgaaactgggtcagcgtctgtgctacctgagcgcgatgaccagcttctttttcgcgatcccgcgtgtgatttttctggcgagcccgctggcgttcctgtttttcagccaaaacattatcgcggcgagcccgctggcggttggcgtgtatgcgatcccgcacatgtttcacagcattgcgaccgcggcgaaagttaacaagggttggcgttacagcttttggagcgaggtttatgaaaccgtgatggcgctgttcctggttcgtgtgaccatcgtgaccatgctgtttccgagcaagggcaagttcaacgttaccgagaaaggtggcgtgctggaacgtgaggaatttgatctgaccgcgacctacccgaacatcattttcgcgatcattatggcgctgggtctgctgcgtggcctgtacgcgctgatcttccagcacctggacatcattagcgagcgtgcgtatgcgctgaactgcatctggagcgttattagcctgatcattctgatggcggttatcagcgtgggtcgtgaaaccaagcagctgcgtcaaagccaccgtatcgaagcgcagattccggttaccgtgtacgactatgatggcaacagcagccacggtattaccgaggatgttagcatgggtggcgtggcgatccacctgccgtggcgtgaagttaccccggatcacccggtgcaagtggttattcacgcggttctggacggcgaggaaatgaacctgccggcgaccatgatccgtagcgcgcagggcaaggcggtgtttacctggagcatcagcaacattcaagttgaggcggcggtggttcgttttgtgttcggtcgtgcggatgcgtggctgcagtggaacaactacgaggacgaccgtccgctgcgtagcctgtggagcctgatcctgagcattaaagcgctgttccgtcgtaagggtcaaatgatcgcgcacagccgtccgaagaaaaagccgattgcgctgccggtggaacgtcgtgaaccgaccaccagccaaggcggccaaaaacaagaaggtaaaatcagccgtgcggcgagctaa

SEQ ID NO: 7 - DNA, 35 bp, Artificial Sequence
  Synthetic bcsA F-primer
aggccttcatatgatgtcagaggttcagtcgtcag

SEQ ID NO: 8 - DNA, 34 bp, Artificial Sequence
  Synthetic bcsA R-primer
aaggccttgagctcttacgaggccgcacgactga

SEQ ID NO: 9 - DNA, 42 bp, Artificial Sequence
  Synthetic GFP F-primer
aaggccttgcggccgcatgagcaaaggagaagaacttttcac

SEQ ID NO: 10 - DNA, 42 bp, Artificial Sequence
  Synthetic GFP R-primer
aaggccttgcggccgcttatttgtagagctcatccatgccat

SEQ ID NO: 11 - DNA, 67 bp, Artificial Sequence
  Synthetic bcsA F-primer
aaggcctttctagaaataattttgtttaactttaagaaggagatataatgtcagaggttcagtcgcc

SEQ ID NO: 12 - DNA, 34 bp, Artificial Sequence
  Synthetic bcsA R-primer
aaggccttgcggccgctcacgaggccgcacggct

SEQ ID NO: 13 - DNA, 2415 bp, Komagataeibacter xylinus
atgaaaatggtgtccctgatcgcgctgctggttttcgcaacgggagcgcaggctgccccgattgcgtccaaagcgccagcccaccagcctacgggcagtgatctcccccccctgcctgcagcggcaccggtggcgccagcggcgcaaccttccgcacaggcggttgatccggcatcagccgcgcccgcgtccgatgcgggaagcgccagcaatgcggatgcgatactggacaatgccgaaaatgcggcaggcgtcggtaccgatgttgcaaccgtccatacctattcccttcaggaactgggtgcgcagagtgcattgaccatgcgcggcgccgccccgctgcaggggttgcagttcgggattccggcagaccagctggtgacatcggcccgactggtcgtgtccggggccatgtcgcccaacctccagcccgacaacagcgcggtgacgatcacgctgaacgaacagtatatcggcacgctgcgtcccgacccgacgcatccggcgttcgggccgctgtcatttgacatcaatccgattttctttgtcagcggcaaccgcctgaacttcaacttcgcttcaggttccaaagggtgcgcggacccgaccaacgggctgcagtgggccagcgtgtctgaacattcgcagctgcagatcacgaccattccgcttcctccccgtcgtcagctggcccgtctgccccagccgttctttgataagactgtaaggcagaaagtcgtcattccgttcgtccttgcacagacatttgatccagaagtgctcaaggcttccggcatcatcgcgtcgtggttcgggcagcagaccgacttccgtggggtcaatttccccgtcttctccaccattccccagaccggcaatgccattgtggtcggcgtggcggatgaactgcctgcagcgctgggccgcccgtccgtcagtggccccaccctgatggaggtcgccaacccatccgatcccaacggcacggtgctgctggtgacggggcgtgaccgcgatgaagtcattaccgccagcaaggggatcggcttcgggtccagcgccctgccggtcgccagccgcatggatgtggcgccgattgatgtcgccccgcgtctggccaatgacgcgccgtccttcattcccaccagccgcccggtgcggctgggtgaactggtgccggtcagcgccctgcagggcgaagggtatacgccgggcgtgctgtcggttccgttccgcgtgtcgcctgacctctatacgtggcgtgaccgtccgtacaagctgaacgtgcgtttccgcgcgccggatggcccgatccttgatgtggcgcgctcgcatctggatgtcggcatcaacaatacctacctgcagtcctattcactgcgggagcagagctcggttgtcgatcagctgctgcgtcgtgttggcgtgggcacccagaacgcgggcgtggagcagcatacgctgaccattccgccgtggatggtgttcggtcaggatcagctgcagttctattttgacgcagccccgctggcacagcccggctgccgtcccggtccgagcctgatccacatgtcggtcgatccggattcgaccattgacctgtccaatgcctatcacatcacgcgcatgcccaacctggcctacatggccagtgcggggtatccgttcacgacctacgccgacctgtcgcgttcggcggtggtgctgccggatcatcccaatggtacggtggtcagcgcgtatctcgacctcatgggcttcatgggggcgacgacatggtatcccgtttcgggcgttgatatcgtttcggccgaccatgtcagcgacgtggcggaccggaacctgattgtcctgtccaccctgtccaacagtgcggatgtatctgccctgctggccaactcggcataccagatttcggatgggcggcttcacatggggctgcgttccaccctgagcggcgtgtggaacatcttccaggatccgatgtcggttatgagcaacacgcacccgaccgaggtcgaaaccacgctgagcggtggcgtcggcgcgatggtggaggcggaatcgccattggcgtccggccgcaccgtgctggccctgctgtcgggtgacgggcaggggcttgataatctggtccagatcctggggcagcgtaagaaccaggcgaaagtacagggtgaccttgtgctggcgcatggtgacgacctgacatcctaccgcagttcgccgctgtatacggttggcacggtgccgctgtggctgattcccgactggtatatgcataaccatcccttccgcgtgatcgtggtcgggctggttggctgtctgctggtggtggctgtcctggtgcgtgccctgttccgccacgcgatgttccgtcgccggcagttgcaggaagaaaggcagaaatcgtga

SEQ ID NO: 14 - DNA, 747 bp, Thermotoga maritima
atgaaggtctctggtggcgaagttcctccattcttcaaggagggtttcttttttacgtccgaagagctgacgcatctgatcaacgtctgcagctctacctctgcaatcatctctgtcctgaaggatagcaagtaccgtcgtagcctggttctgtacctgaaacgtccgctgtctaaagacgctctgctgctgattcagactctgctgactactctggaacgtgaagatctgtctttcggtgtgaaagagctggagtacatggcataccacgacccgctgaccggtctgccgaatcgtcgttatttcttcgaactgggtaaccgttacctggatctggctaaacgtgagggtaaaaaagtgttcgtgctgttcgtggacctggctggtttcaaagcgatcaacgatacctacggtcacctgtctggcgatgaagtgctgaaaaccgtttccaaacgtatcctggaccgtgttcgtcgtagcgacgtagtagcccgctatggcggcgacgaatttaccattctgctgtatgacatgaaagaagaatacctgaaatccctgctggaacgcatcctgtccaccttccgcgaaccggtacgcgttgaaaacaaacacctgtccgtaaccccgaacattggcgttgcccgctttccggaagacggcgaaaacctggaagaactgctgaaagttgcggatatgcgcatgtacaaagcgaaagaaatgaaagttccgtatttcagcctgtcctaa

SEQ ID NO: 15 - DNA, 2265 bp, Komagataeibacter xylinus
atgtcagaggttcagtcgccagtacccgcggagagtaggctagaccgcttttccaacaagatactgtcactgcgtggggccaactatatagttggagcgctggggctttgtgcacttatcgccgcaaccacggttacgctgtccattaatgagcagctgattgtggcacttgtgtgtgtgctcgtctttttcattgtcgggcggggcaagagccggcgtacccagatctttctcgaggtgctctcggcgctggtttccctgcgttacctgacatggcgcctgaccgaaacgctggacttcgacacatggattcagggcgggctgggtgtgaccctgctcatcgccgagctgtatgccctgtacatgctgtttctcagctatttccagacaatccagccgcttcatcgcacgccgctccccctgccggacaatgttgatgactggcccaccgtcgatatcttcatcccgacctatgatgaacagctgagcatcgtgcgcctgaccgtgctgggcgcgctgggtatcgactggccacccgataaagtgaatgtctatatccttgatgatggcgtgcgcccggaattcgaacagtttgccagggaatgtggttccctttacatcgggcgcgtggacagttcgcacgccaaggcgggtaacctaaaccacgccattaagcagacaagcggcgattacatcctcatcctggattgtgaccatattccgacacgcgcgttcctgcagatcgcgatgggctggatggtcgccgaccgcaagattgccctgatgcagacgccgcatcacttctactcccccgatccgttccagcgtaacctcgccgtgggatatcgcaccccgccggaaggcaacctgttctacggcgtcattcaggatggtaacgacttctgggatgccaccttcttctgcggctcgtgcgccatcctgcgccgtgaggcgattgaatcgatcggcggcttcgcggttgaaaccgtgacggaagatgcccataccgccctgcgcatgcagcgccgtggctggtccactgcctacttgcgcattcctgtggccagtggcctggctaccgagcgcctgacaacccatatcggccagcgcatgcgctgggcgcgcggcatgatccagatcttccgcgtggataatccgatgcttgggtcggggctgaagcttggccagcggctgtgctacctctcggctatgacgtcgttcttcttcgccattccgcgcgtcatcttcctcgcctcgccgctggcgttcctgttcgcgggccagaacatcatcgccgcctcgccgctggccgttctggcctacgccattccgcatatgttccactccatcgcgaccgccgccaaggtaaacaagggctggcgctactcgttctggagtgaagtgtacgaaaccaccatggcgctgttcctggtgcgcgtgaccatcatcaccatgatgttcccctctaagggcaagttcaacgtgacggaaaagggtggggtgctggaggaggaagagttcgatcttggcgcgacctaccccaacatcatctttgccgtcatcatggcgcttggcctgctgatcggcctgttcgaactgatcttccacttcagccagcttgatggcatcgccatgcgcgcctacgcgctgaactgcatctgggccgcgatcagtctcatcatccttctggctgccattgcggtggggcgtgaaaccaaacaggtccgttacagccatcgtatcgatgcgcatatcccggtaacggtttatgaagcgccggtcgcggggcagcccaatacctaccataatgcgacaccgggcatgacccaggatgtttccatgggtggtgttgccgtgcatatgccctggcccgatatcggctcggggccggtcaagacacgtatccatgccgtgctcgatggcgaggagatcgatattcccgccaccatgctgcgctgcaagaatggcaaggccgtgttcacatgggacaataatgaccttgatacggaacgcgatatcgtccgcttcgtgttcgggcgggccgatgcctggctgcaatggaataattatgaggatgacaggccgctacgcagcctgtggagcctgctgctcagcattaaggcgctgttccgcaaaaaaggcaaaatgatggccaatagtcgtccaacaaaaaaaccacgtgcactaccggttgagcgcagggagcccacaaccatccagagtggacagacacaggaaggaaagatcagccgtgcggcctcgtga

SEQ ID NO: 16 - DNA, 535 bp, Komagataeibacter xylinus
aacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaaatgtcagaccttacggagaaaataagaaa

SEQ ID NO: 17 - DNA, 40 bp, Artificial Sequence
  Synthetic nucleotide sequence that encodes a cellulose synthase  A
cctgggtgtcacgctgctgctggcggaactgtatgcgctc

SEQ ID NO: 18 - protein, 13 aa, Artificial Sequence
  Synthetic cellulose synthase A
LGVTLLLAELYAL

SEQ ID NO: 19 - DNA, 40 bp, Artificial Sequence
  Synthetic a nucleotide sequence that encodes a cellulose synthase A
tctgggcgttaccttgttactggcggaactgtacgcgctg

SEQ ID NO: 20 - DNA, 2238 bp, Artificial Sequence
  Synthetic Komagataeibacter xylinus modified
atgtcagaggttcagtcgtcagcgcctgcggaaagctggttcggccgcttttccaacaagatactgtcactgcgcggtgccagctatgtcgttggggcgttggggctttgcgccctgcttgccgcaaccatggttacgctgtcgcttaatgaacagatgattgtggcattagtgtgtgtggcggtgttttttatcgtcggccgccgcaaaagccgtcgcacccaggtctttctggaggtgctgtcggcgctggtgtccctgcggtatctgacgtggcggctgacggaaacgctggactttgatacctggacgcagggcatcctgggtgtcacgttgctgctggcggaactgtatgcgctctacatgctgttcctcagttatttccagacgatttcccccctgcatcgtgcgccgctgccgctgccggccaatcccgatgagtggcccacggttgatattttcatcccgacctatgacgaagcactgagcatcgtgcgtctgacggtgctcggagcgttgggtatcgactggccgcctgataaggtgaacgtctatattctggatgacggcaggcgtgaggaattcgcccgttttgccgaggcctgcggcgcgcgttacatcgcccgtcccgataacgcgcacgccaaggccggtaacctgaactacgccattaaacataccacgggcgatcacatcctcatcctggactgtgaccatatcccgacgcgtgctttcctgcagatctccatgggctggatggttagcgattcgaacatcgccctgctgcagacgccgcatcacttctattcccccgatccgttccagcgtaacctggcggtggggtaccgcaccccgcccgaaggcaatctgttctatggcgtcattcaggacggtaacgacttctgggatgcgaccttcttctgcggatcgtgcgccatcctgcgccgtaaggcgattgaggaaatcggcggcttcgcaaccgaaaccgtgacagaagacgcccataccgcgctgcgtatgcagcgcaagggctggtcgaccgcctacctgcgcattccgctggccagtggtctggcgacagaacgtctcattacgcatatcgggcagcgtatgcgctgggcccgtggcatgatccagattttccgcgttgataacccgatgcttggctcgggtctgaagcttgggcagcgtctttgctacctgtcggccatgacgtcgttcttcttcgccattccgcgcgtcatcttccttgcatccccgctggccttcctgttcttcagccagaatatcatcgcggcatccccgctggcggtgggggtctacgccatcccgcacatgttccattccattgcgactgcggcgaaggtcaacaagggctggcggtattcgttctggagtgaagtgtacgaaaccgtcatggcgctgttcctggtgcgggtgaccatcgtcacgatgctgttcccctccaagggcaagttcaacgtgacggaaaaaggtggcgttctggaacgtgaggaattcgacctcaccgccacctatccgaatattattttcgccatcatcatggccctcggcctgttgcgtgggctatatgcgctgatcttccagcacctggacatcatttcggaacgtgcgtacgcgctgaactgcatctggtcggtgatcagtctgatcatcctgatggcggcaatctccgtggggcgtgaaacaaagcagctgcgccagagccatcgtatcgaagcccagatccccgttacggtttatgattacgatggcaattcgagccacggcattaccgaagacgtctccatgggcggtgtggcgatccacctgccatggcgtgaggttactcccgatcaccctgtacaggtcgtgatccatgccgtactggatggcgaggagatgaaccttccggccaccatgatccgcagtgcccagggcaaggcggtgtttacgtggtcgatcagtaacattcaggttgaggcggccgtggtccggtttgtgttcggacgcgccgatgcctggctgcagtggaataattatgaggatgaccggccgttacgaagcctgtggagtctgatcctcagcatcaaggcactgttccgcaggaagggtcagatgattgcccatagtcgtcccaaaaagaaaccaattgcactgccggttgagcgtagggagccaacaaccagccagggtggtcagaaacaggaaggaaagatcagtcgtgcggcctcgtga

SEQ ID NO: 21 - DNA, 2238 bp, Artificial Sequence
  Synthetic Komagataeibacter xylinus modified
atgtcagaggttcagtcgtcagcgcctgcggaaagctggttcggccgcttttccaacaagatactgtcactgcgcggtgccagctatgtcgttggggcgttggggctttgcgccctgcttgccgcaaccatggttacgctgtcgcttaatgaacagatgattgtggcattagtgtgtgtggcggtgttttttatcgtcggccgccgcaaaagccgtcgcacccaggtctttctggaggtgctgtcggcgctggtgtccctgcggtatctgacgtggcggctgacggaaacgctggactttgatacctggacgcagggcatcctgggtgtcacgctgttactggcggaactgtatgcgctctacatgctgttcctcagttatttccagacgatttcccccctgcatcgtgcgccgctgccgctgccggccaatcccgatgagtggcccacggttgatattttcatcccgacctatgacgaagcactgagcatcgtgcgtctgacggtgctcggagcgttgggtatcgactggccgcctgataaggtgaacgtctatattctggatgacggcaggcgtgaggaattcgcccgttttgccgaggcctgcggcgcgcgttacatcgcccgtcccgataacgcgcacgccaaggccggtaacctgaactacgccattaaacataccacgggcgatcacatcctcatcctggactgtgaccatatcccgacgcgtgctttcctgcagatctccatgggctggatggttagcgattcgaacatcgccctgctgcagacgccgcatcacttctattcccccgatccgttccagcgtaacctggcggtggggtaccgcaccccgcccgaaggcaatctgttctatggcgtcattcaggacggtaacgacttctgggatgcgaccttcttctgcggatcgtgcgccatcctgcgccgtaaggcgattgaggaaatcggcggcttcgcaaccgaaaccgtgacagaagacgcccataccgcgctgcgtatgcagcgcaagggctggtcgaccgcctacctgcgcattccgctggccagtggtctggcgacagaacgtctcattacgcatatcgggcagcgtatgcgctgggcccgtggcatgatccagattttccgcgttgataacccgatgcttggctcgggtctgaagcttgggcagcgtctttgctacctgtcggccatgacgtcgttcttcttcgccattccgcgcgtcatcttccttgcatccccgctggccttcctgttcttcagccagaatatcatcgcggcatccccgctggcggtgggggtctacgccatcccgcacatgttccattccattgcgactgcggcgaaggtcaacaagggctggcggtattcgttctggagtgaagtgtacgaaaccgtcatggcgctgttcctggtgcgggtgaccatcgtcacgatgctgttcccctccaagggcaagttcaacgtgacggaaaaaggtggcgttctggaacgtgaggaattcgacctcaccgccacctatccgaatattattttcgccatcatcatggccctcggcctgttgcgtgggctatatgcgctgatcttccagcacctggacatcatttcggaacgtgcgtacgcgctgaactgcatctggtcggtgatcagtctgatcatcctgatggcggcaatctccgtggggcgtgaaacaaagcagctgcgccagagccatcgtatcgaagcccagatccccgttacggtttatgattacgatggcaattcgagccacggcattaccgaagacgtctccatgggcggtgtggcgatccacctgccatggcgtgaggttactcccgatcaccctgtacaggtcgtgatccatgccgtactggatggcgaggagatgaaccttccggccaccatgatccgcagtgcccagggcaaggcggtgtttacgtggtcgatcagtaacattcaggttgaggcggccgtggtccggtttgtgttcggacgcgccgatgcctggctgcagtggaataattatgaggatgaccggccgttacgaagcctgtggagtctgatcctcagcatcaaggcactgttccgcaggaagggtcagatgattgcccatagtcgtcccaaaaagaaaccaattgcactgccggttgagcgtagggagccaacaaccagccagggtggtcagaaacaggaaggaaagatcagtcgtgcggcctcgtga

SEQ ID NO: 22 - DNA, 2238 bp, Artificial Sequence
  Synthetic Komagataeibacter xylinus modified
atgtcagaggttcagtcgtcagcgcctgcggaaagctggttcggccgcttttccaacaagatactgtcactgcgcggtgccagctatgtcgttggggcgttggggctttgcgccctgcttgccgcaaccatggttacgctgtcgcttaatgaacagatgattgtggcattagtgtgtgtggcggtgttttttatcgtcggccgccgcaaaagccgtcgcacccaggtctttctggaggtgctgtcggcgctggtgtccctgcggtatctgacgtggcggctgacggaaacgctggactttgatacctggacgcagggcatcctgggtgtcacgctgttgctggcggaactgtatgcgctctacatgctgttcctcagttatttccagacgatttcccccctgcatcgtgcgccgctgccgctgccggccaatcccgatgagtggcccacggttgatattttcatcccgacctatgacgaagcactgagcatcgtgcgtctgacggtgctcggagcgttgggtatcgactggccgcctgataaggtgaacgtctatattctggatgacggcaggcgtgaggaattcgcccgttttgccgaggcctgcggcgcgcgttacatcgcccgtcccgataacgcgcacgccaaggccggtaacctgaactacgccattaaacataccacgggcgatcacatcctcatcctggactgtgaccatatcccgacgcgtgctttcctgcagatctccatgggctggatggttagcgattcgaacatcgccctgctgcagacgccgcatcacttctattcccccgatccgttccagcgtaacctggcggtggggtaccgcaccccgcccgaaggcaatctgttctatggcgtcattcaggacggtaacgacttctgggatgcgaccttcttctgcggatcgtgcgccatcctgcgccgtaaggcgattgaggaaatcggcggcttcgcaaccgaaaccgtgacagaagacgcccataccgcgctgcgtatgcagcgcaagggctggtcgaccgcctacctgcgcattccgctggccagtggtctggcgacagaacgtctcattacgcatatcgggcagcgtatgcgctgggcccgtggcatgatccagattttccgcgttgataacccgatgcttggctcgggtctgaagcttgggcagcgtctttgctacctgtcggccatgacgtcgttcttcttcgccattccgcgcgtcatcttccttgcatccccgctggccttcctgttcttcagccagaatatcatcgcggcatccccgctggcggtgggggtctacgccatcccgcacatgttccattccattgcgactgcggcgaaggtcaacaagggctggcggtattcgttctggagtgaagtgtacgaaaccgtcatggcgctgttcctggtgcgggtgaccatcgtcacgatgctgttcccctccaagggcaagttcaacgtgacggaaaaaggtggcgttctggaacgtgaggaattcgacctcaccgccacctatccgaatattattttcgccatcatcatggccctcggcctgttgcgtgggctatatgcgctgatcttccagcacctggacatcatttcggaacgtgcgtacgcgctgaactgcatctggtcggtgatcagtctgatcatcctgatggcggcaatctccgtggggcgtgaaacaaagcagctgcgccagagccatcgtatcgaagcccagatccccgttacggtttatgattacgatggcaattcgagccacggcattaccgaagacgtctccatgggcggtgtggcgatccacctgccatggcgtgaggttactcccgatcaccctgtacaggtcgtgatccatgccgtactggatggcgaggagatgaaccttccggccaccatgatccgcagtgcccagggcaaggcggtgtttacgtggtcgatcagtaacattcaggttgaggcggccgtggtccggtttgtgttcggacgcgccgatgcctggctgcagtggaataattatgaggatgaccggccgttacgaagcctgtggagtctgatcctcagcatcaaggcactgttccgcaggaagggtcagatgattgcccatagtcgtcccaaaaagaaaccaattgcactgccggttgagcgtagggagccaacaaccagccagggtggtcagaaacaggaaggaaagatcagtcgtgcggcctcgtga

SEQ ID NO: 23 - DNA, 2238 bp, Artificial Sequence
  Synthetic Komagataeibacter xylinus modified
atgtcagaggttcagtcgtcagcgcctgcggaaagctggttcggccgcttttccaacaagatactgtcactgcgcggtgccagctatgtcgttggggcgttggggctttgcgccctgcttgccgcaaccatggttacgctgtcgcttaatgaacagatgattgtggcattagtgtgtgtggcggtgttttttatcgtcggccgccgcaaaagccgtcgcacccaggtctttctggaggtgctgtcggcgctggtgtccctgcggtatctgacgtggcggctgacggaaacgctggactttgatacctggacgcagggcatcctgggtgtcacgctgctactggcggaactgtatgcgctctacatgctgttcctcagttatttccagacgatttcccccctgcatcgtgcgccgctgccgctgccggccaatcccgatgagtggcccacggttgatattttcatcccgacctatgacgaagcactgagcatcgtgcgtctgacggtgctcggagcgttgggtatcgactggccgcctgataaggtgaacgtctatattctggatgacggcaggcgtgaggaattcgcccgttttgccgaggcctgcggcgcgcgttacatcgcccgtcccgataacgcgcacgccaaggccggtaacctgaactacgccattaaacataccacgggcgatcacatcctcatcctggactgtgaccatatcccgacgcgtgctttcctgcagatctccatgggctggatggttagcgattcgaacatcgccctgctgcagacgccgcatcacttctattcccccgatccgttccagcgtaacctggcggtggggtaccgcaccccgcccgaaggcaatctgttctatggcgtcattcaggacggtaacgacttctgggatgcgaccttcttctgcggatcgtgcgccatcctgcgccgtaaggcgattgaggaaatcggcggcttcgcaaccgaaaccgtgacagaagacgcccataccgcgctgcgtatgcagcgcaagggctggtcgaccgcctacctgcgcattccgctggccagtggtctggcgacagaacgtctcattacgcatatcgggcagcgtatgcgctgggcccgtggcatgatccagattttccgcgttgataacccgatgcttggctcgggtctgaagcttgggcagcgtctttgctacctgtcggccatgacgtcgttcttcttcgccattccgcgcgtcatcttccttgcatccccgctggccttcctgttcttcagccagaatatcatcgcggcatccccgctggcggtgggggtctacgccatcccgcacatgttccattccattgcgactgcggcgaaggtcaacaagggctggcggtattcgttctggagtgaagtgtacgaaaccgtcatggcgctgttcctggtgcgggtgaccatcgtcacgatgctgttcccctccaagggcaagttcaacgtgacggaaaaaggtggcgttctggaacgtgaggaattcgacctcaccgccacctatccgaatattattttcgccatcatcatggccctcggcctgttgcgtgggctatatgcgctgatcttccagcacctggacatcatttcggaacgtgcgtacgcgctgaactgcatctggtcggtgatcagtctgatcatcctgatggcggcaatctccgtggggcgtgaaacaaagcagctgcgccagagccatcgtatcgaagcccagatccccgttacggtttatgattacgatggcaattcgagccacggcattaccgaagacgtctccatgggcggtgtggcgatccacctgccatggcgtgaggttactcccgatcaccctgtacaggtcgtgatccatgccgtactggatggcgaggagatgaaccttccggccaccatgatccgcagtgcccagggcaaggcggtgtttacgtggtcgatcagtaacattcaggttgaggcggccgtggtccggtttgtgttcggacgcgccgatgcctggctgcagtggaataattatgaggatgaccggccgttacgaagcctgtggagtctgatcctcagcatcaaggcactgttccgcaggaagggtcagatgattgcccatagtcgtcccaaaaagaaaccaattgcactgccggttgagcgtagggagccaacaaccagccagggtggtcagaaacaggaaggaaagatcagtcgtgcggcctcgtga

(19) United States
(12) Patent Application Publication
(10) Pub. No.: US 2018/0148696 A1
(43) Pub. Date: May 31, 2018

(54) MICROORGANISM INCLUDING GENETIC MODIFICATION THAT INCREASES ACTIVITY OF CELLULOSE SYNTHASE AND METHOD FOR PRODUCING CELLULOSE BY USING THE SAME

(71) Applicants: Samsung Electronics Co., Ltd., Suwon-si (KR); Korea Advanced Institute of Science and Technology, Daejeon (KR)

(72) Inventors: Jinsuk Lee, Seoul (KR); Kijun Jeong, Daejeon (KR); Jaehyung Lee, Daejeon (KR); Jiae Yun, Hwaseong-si (KR); Jinkyu Kang, Hwaseong-si (KR); Goun Kim, Goyang-si (KR); Jinhwan Park, Suwon-si (KR)

(21) Appl. No.: 15/826,187

(22) Filed: Nov. 29, 2017

(30) Foreign Application Priority Data
Nov. 29, 2016 (KR) 10-2016-0160784

(51) Int. Cl. C12N 9/10; C12P 19/04

(52) U.S. Cl. CPC C12N 9/1059; C12P 19/04; C12Y 204/01012

(57) ABSTRACT

Provided are a recombinant microorganism including a genetic modification that increases activity of a cellulose synthase, a gene that encodes the cellulose synthase having increased activity, and a method of producing cellulose by using the recombinant microorganism.

### CROSS-REFERENCE TO RELATED APPLICATION

This application claims the benefit of Korean Patent Application No. 10-2016-0160784, filed on Nov. 29, 2016, in the Korean Intellectual Property Office, the disclosure of which is incorporated herein in its entirety by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 38,824 Byte ASCII (Text) file named “733300_ST25.TXT,” created on Nov. 29, 2017.

### BACKGROUND

### 1. Field

The present disclosure relates to a recombinant microorganism including a genetic modification that increases activity of a cellulose synthase, a gene that encodes the cellulose synthase having increased activity, the cellulose synthase having increased activity, and a method of producing cellulose by using the recombinant microorganism.

### 2. Description of the Related Art

In cellulose produced by cultivating microorganisms, also known as microbial cellulose, glucose may be present in the form of β-1,4 glucan as a primary structure, which forms a network structure of fibril bundles.

Microbial cellulose is 100 nm or less in width, and, unlike plant cellulose, is free of lignin nor hemicelluloses. Additionally, compared to plant cellulose, microbial has improved wetting properties, improved hygroscopic properties, higher strength, higher elasticity, and higher heat resistance. Due to these properties, microbial cellulose has been developed for applications in various industrial fields, including cosmetics, medicine, dietary fibers, vibration plates for sound systems, and functional films.

Therefore, there is a need to develop new microorganisms and methods to increase the production of microbial cellulose. This invention provides such microorganisms and methods.

### SUMMARY

Provided is a recombinant microorganism including a genetic modification that increases activity of a cellulose synthase.

Also provided is a method of producing cellulose the method including culturing, a recombinant microorganism having a genetic modification that increases activity of a cellulose synthase in a culture medium; and separating cellulose from the culture medium.

Further provided is a polynucleotide encoding a cellulose synthase having an activity belonging to EC 2.4.1.12, the polynucleotide having at least one nucleotide substitution of nucleotides corresponding to positions 334 to 351 in a nucleotide sequence of SEQ ID NO: 2.

### BRIEF DESCRIPTION OF THE DRAWINGS

These and/or other aspects will become apparent and more readily appreciated from the following description of the embodiments, taken in conjunction with the accompanying drawings in which:

FIG. 1A illustrates the structure of an expression vector including a T7 promoter, a cellulose synthase A gene fragment, and a green fluorescent protein (GFP)-tag;

FIG. 1B illustrates fragments (truncated forms) of various lengths of the cellulose synthase A gene;

FIG. 2 illustrates the expression level of the various cellulose synthase A gene fragments;

FIG. 3A illustrates cellulose synthase A gene fragments of 333nt, 336nt, 339nt, 342nt, 345nt, 348nt, and 351nt, respectively;

FIG. 3B illustrates the expression level of the cellulose synthase A gene in cells transformed with the vector pET22b-bcsA (E25, 333nt)-GFP, pET22b-bcsA (E25, 336nt)-GFP, pET22b-bcsA (E25, 339nt)-GFP, pET22b-bcsA (E25, 342nt)-GFP, pET22b-bcsA (E25, 345nt)-GFP, pET22b-bcsA (E25, 348nt)-GFP, or pET22b-bcsA (E25, 351nt)-GFP

FIG. 4A illustrates the expression level of the cellulose synthase A gene in cells transformed with the expression vector pET22b-bcsA (E25, 333nt)-GFP, pET22b-bcsA (E25, 336nt)-GFP, pET22b-bcsA (E25, 339nt)-GFP, pET22b-bcsA (E25, 342nt)-GFP, pET22b-bcsA (E25, 345nt)-GFP, pET22b-bcsA (E25, 348nt)-GFP, pET22b-bcsA (E25, 351nt)-GFP, or pET22b-bcsA (E25, 351nt, 334-336, 337-339 mod)-GFP;

FIG. 4B illustrates the expression level of the cellulose synthase A gene in cells (transformed with the expression vector pET22b-bcsA (E25, 333nt)-GFP, pET22b-bcsA (E25, 336nt)-GFP, pET22b-bcsA (E25, 336nt, 334-336 mod)-GFP, pET22b-bcsA (E25, 339nt, 334-336, 337-339 mod)-GFP, pET22b-bcsA (E25, 351nt)-GFP, or pET22b-bcsA (E25, 351nt, 334-336, 337-339 mod)-GFP;

FIG. 5A illustrates the nucleotide sequence (SEQ ID NO: 17) and corresponding amino acid sequence (SEQ ID NO: 18) that regulates the expression of the cellulose synthase A gene of K. xylinus E25;

FIG. 5B illustrates a nucleotide sequence (SEQ ID NO: 19) and corresponding amino acid sequence (SEQ ID NO: 18) that encodes a cellulose synthase A having increased activity;

FIG. 6 illustrates the expression level of the cellulose synthase A gene in cells transformed with the expression vector pET22b-bcsA (41431, 333nt)-GFP, pET22b-bcsA (41431, 336nt)-GFP, pET22b-bcsA (41431, 339nt)-GFP, pET22b-bcsA (41431, 342nt)-GFP, pET22b-bcsA (41431, 345nt)-GFP, pET22b-bcsA (41431, 348nt)-GFP, or pET22b-bcsA (41431, 351nt)-GFP;

FIG. 7A illustrates the yield of cellulose nanofiber (CNF) after incubation of E. coli RP/pET-bcsA(E25, 2238nt)B+pACYC-DGC, E. coli RP/pET-bcsA(E25, 2238nt, 334-336, 337-339 mod)B+pACYC-DGC, and E. coli RP strains in methyl red (MR) media; and

FIG. 7B illustrates the yield of CNF after incubation of E. coli RP/pET-bcsA(E25, 2238nt)B+pACYC-DGC, E. coli RP/pET-bcsA(E25, 2238nt, 334-336, 337-339 mod)B+pACYC-DGC, and E. coli RP strains in Luria-Bertani (LB) media.

### DETAILED DESCRIPTION

The terms “increase in activity”, or “increased activity” or like terms, as used herein refers to a detectable increase in the activity level of a cell, protein, or enzyme relative to the activity of a cell protein, or enzyme of the same type, that does not have a given genetic modification (e.g., a parent cell or a native, original, or “wild-type” cell, protein, or enzyme). For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more relative to the activity of a cell, protein, or enzyme of the same type (e.g., a wild-type cell, protein, or enzyme) that does not have a given modification or has not been engineered. A cell including a protein or enzyme having increased enzymatic activity may be verified by any methods known in this art.

A cell having increased activity of an enzyme or polypeptide may be induced by increasing expression or specific activity of the enzyme or polypeptide. The increase in expression may be achieved by introduction of a polynucleotide that encodes the enzyme or polypeptide into cells, by increasing the copy number of the polynucleotide that encodes the enzyme or polypeptide in a cell, or by a mutation of a regulatory region of the polynucleotide. The introduction may be a transient introduction in which the gene is not integrated into a genome, or an introduction that results in integration of the gene into the genome. The introduction may be performed, for example, by introducing a vector comprising a polynucleotide encoding the enzyme or polypeptide into the cell.

The polynucleotide may be operably linked to a regulatory sequence that enables expression of the enzyme or polypeptide, for example, a promoter, an enhancer, a polyadenylation site, or a combination thereof. The polynucleotide may be endogenous or exogenous to the microorganism in which it is inserted. As used herein, an endogenous gene refers to a polynucleotide that is present in intrinsic genetic material of the microorganism prior to a given genetic manipulation, for instance in the genetic material of the wild-type or native microorganism. As used herein, an exogenous gene refers to a polynucleotide introduced into cells from outside, and may be homologous or heterologous with respect to the host cell into which the polynucleotide is introduced. The term “homologous” means “native” to a given species, and “heterologous” means “foreign” or not “native” to the species.

An increase in copy number of a polynucleotide refers to any increase in copy number. For example, an increase in copy number may be from the introduction of an exogenous polynucleotide or amplification of an endogenous polynucleotide, and includes the introduction of a heterologous polynucleotide that is not present in a non-engineered cell or parent cell. The introduction of a polynucleotide may be transient introduction of the polynucleotide, lacking integration into the genome of the cell, or may be insertion of the polynucleotide into the genome. The introduction may be achieved, for example, through introduction of a vector into the cell, the vector including a polynucleotide encoding a target polypeptide, and then the vector being copied in the cell or the polynucleotide being integrated into the genome.

The introduction of a gene may be achieved by any method known in the art, for example, transformation, transfection, or electroporation.

The term “vehicle” or “vector” as used herein refers to a nucleic acid molecule that may deliver nucleic acids linked thereto. The vector may include, for example, a plasmid expression vector, a virus expression vector, for example, a replication defective retroviral vector, an adenoviral vector, or an adeno-associated viral vector.

The polynucleotide as used herein may be engineered or manipulated by any molecular biological method known in the art.

The term “parent cell” as used herein refers to an original cell, for example, a non-genetically engineered cell of the same type as an engineered cell. Regarding a particular genetic modification, the “parent cell” may be a cell that does not have the particular genetic modification. Accordingly, the parent cell may be a cell that is used as a starting material for the production of a genetically engineered microorganism including a protein having increased activity. The same comparison may apply to other types of genetic modification.

The terms “gene” and “polynucleotide” as used herein are synonymous and refer to a nucleic acid fragment that encodes a particular protein, and may optionally include at least one regulatory sequence of a 5′-non-coding sequence and a 3′-non-coding sequence.

The term “sequence identity” of a nucleic acid or polypeptide as used herein refers to a degree of identity of bases or amino acid residues of two corresponding sequences over a particular region measured after the sequences are aligned to be matched with each other as much as possible. The sequence identity is a value that is measured by comparing two optimally aligned corresponding sequences of a particular comparable region, wherein in the comparable region, a part of the sequence may be added or deleted with respect to a reference sequence. In some embodiments, a percentage of the sequence identity may be calculated by comparing two optimally aligned corresponding sequences in an entire comparable region, determining the number of locations where an amino acid or a nucleic acid is identical in the two sequences to obtain the number of matched locations, dividing the number of the matched locations by the total number (that is, a range size) of all locations within a comparable range, and multiplying the result by 100 to obtain a percentage of the sequence identity. The percentage of the sequence identity may be determined by using known sequence comparison programs, examples of which include BLASTN (NCBI) and BLASTP (NCBI), CLC Main Workbench (CLC bio.), and MegAlign™ (DNASTAR Inc.). Unless stated otherwise herein, parameters selected to execute such a program may be as follows: Ktuple=2, Gap Penalty=4, and Gap length penalty=12.

In identifying polypeptides or polynucleotides of different species that may have an identical or similar function or activity, various levels of similarity in sequence identity may be used. For example, similarity may have a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

According to an aspect of the present disclosure, there is provided a recombinant microorganism including a genetic modification that increases activity of a cellulose synthase.

The cellulose synthase may be bacterial cellulose synthase A (bcsA) (also called cellulose synthase A or CesA), a fragment of bcsA comprising an active subunit which induces a catalytic reaction, and/or bacterial cellulose synthase B (bcsB) (also called cellulose synthase B or CesB). The cellulose synthase activity may be dependent on the presence of cyclic-di-GMP as an allosteric activator, wherein diguanyl cyclase (DGC) may synthesize the cyclic-di-GMP. Cellulose synthesis may be facilitated by the generation of cellulose chains from UDP-glucose through polymerization of a cellulose synthase complex including inner membrane-associated cellulose synthase A and cellulose synthase B. By simultaneous expression of the cellulose synthase A, the cellulose synthase B, and the diguanyl cyclase, the synthesis of cellulose may be facilitated.

The cellulose synthase may be a cellulose synthase belonging to EC 2.4.1.12 in the enzyme commission number. The cellulose synthase may be from the genus Komagataeibacter. For example, the cellulose synthase may be from Komagataeibacter xylinus (K. xylinus). For example, the cellulose synthase may be from K. xylinus E25.

In one embodiment, the genetic modification, that increases the activity of a cellulose synthase, is the introduction of a gene encoding a cellulose synthase having an activity belonging to EC 2.4.1.12.

In another embodiment, the genetic modification, that increases the activity of a cellulose synthase, may include an exogenous gene having at least one nucleotide substitution of nucleotides corresponding to positions 334 to 351 in the nucleotide sequence of SEQ ID NO: 2. Thus, for example, the gene can comprise SEQ ID NO: 2 with at least one nucleotide substitution at position 334 to 351. In one embodiment, the gene may include at least one nucleotide substitution of nucleotides corresponding to positions 334 to 339 in the nucleotide sequence of SEQ ID NO: 2. For example, the gene may include a substitution of nucleotides corresponding to positions 334 to 336 in the nucleotide sequence of SEQ ID NO: 2 with TTG, TTA, CTT, CTC, CTA, or a combination thereof (e.g., SEQ ID NO: 20); a substitution of nucleotides corresponding to positions 337 to 339 in the nucleotide sequence of SEQ ID NO: 2 with TTG, TTA, CTT, CTC, CTA, or a combination thereof (e.g., SEQ ID NO: 21, 22, or 23). For example, the gene may include a substitution of nucleotides corresponding to positions 334 to 336 in the nucleotide sequence of SEQ ID NO: 2 with TTG (SEQ ID NO: 20); nucleotides corresponding to positions 337 to 339 in the nucleotide sequence of SEQ ID NO: 2 with TTA (SEQ ID NO: 21), TTG (SEQ ID NO: 22), or CTA (SEQ ID NO: 23); or a combination thereof. In particular, the gene may include a substitution of nucleotides corresponding to positions 334 in the nucleotide sequence of SEQ ID NO: 2 with T, a substitution of nucleotides corresponding to positions 337 in the nucleotide sequence of SEQ ID NO: 2 with T, a substitution of nucleotides corresponding to positions 339 in the nucleotide sequence of SEQ ID NO: 2 with A, or a combination thereof. For example, the gene can comprise SEQ ID NO: 2 with any one or more of the foregoing mutations. The gene may include a nucleotide sequence of which a 334^{nd}, 337^{nd} and 339^{nd} nucleotide sequence of SEQ ID NO: 2 or 6, 15 is substituted with T, T and A, respectively.

The cellulose synthase may be a polypeptide having an amino acid sequence of SEQ ID NO: 1. The cellulose synthase may have a sequence identity with the amino acid sequence of SEQ ID NO: 1 of about 90% or greater, about 95% or greater, about 96% or greater, about 97% or greater, about 98% or greater, or about 99% or greater.

In the recombinant microorganism, the gene encoding cellulose synthase may include a synonymous nucleic acid alteration of a codon encoding an amino acid residue corresponding to position L112, L113, L114, A115, E116, or L117, or a combination thereof, in an amino acid sequence of SEQ ID NO: 1. For example, the gene encoding cellulose synthase may include a synonymous nucleic acid alteration of a codon encoding an amino acid residue corresponding to position L112 or L113, or a combination thereof, in the amino acid sequence of SEQ ID NO: 1.

The “synonymous nucleic acid alteration” is a latent or silent mutation, and may refer to a mutation in a gene that encodes an amino acid sequence of a protein, but does not alter the amino acid sequence of the protein encoded by the gene, and is in other words, a synonymous codon mutation.

The genetic modification may result in a synonymous nucleic acid alteration in which a nucleotide of a codon that encodes an amino acid residue corresponding to position L112, L113, L114, A115, E116, or L117, or a combination thereof, in an amino acid sequence of SEQ ID NO: 1 is substituted with another nucleotide, the substituted nucleotide is transcribed into RNA to form a codon, and then the codon is translated into the same amino acid. This is attributed to the fact that mostly multiple genetic codes (codons) are assigned to one amino acid.

The gene that encodes a polypeptide including the amino acid sequence of SEQ ID NO: 1 may include the nucleotide sequence of SEQ ID NO: 2 or a variant thereof. Accordingly, the gene may include at least one nucleotide substitution of nucleotides corresponding to positions 334 to 351 in the nucleotide sequence of SEQ ID NO: 2, and the cellulose synthase may include a synonymous nucleic acid alteration of a codon encoding an amino acid residue corresponding to position L112, L113, L114, A115, E116, or L117, or a combination thereof, in the amino acid sequence of SEQ ID NO: 1. The gene may include at least one nucleotide substitution of nucleotides corresponding to positions 334 to 339 in the nucleotide sequence of SEQ ID NO: 2, and the gene encoding cellulose synthase may include a synonymous nucleic acid alteration of a codon encoding an amino acid residue corresponding to position L112 or L113, or a combination thereof, in the amino acid sequence of SEQ ID NO: 1. The gene may include a substitution of nucleotides corresponding to positions 334 to 336 in the nucleotide sequence of SEQ ID NO: 2 with TTG, TTA, CTT, CTC, or CTA, a substitution of nucleotides corresponding to positions 337 to 339 of SEQ ID NO: 2 with TTG, TTA, CTT, CTC, or CTA, or a combination thereof; and the gene encoding cellulose synthase may include a synonymous nucleic acid alteration of a codon encoding an amino acid residue corresponding to position L112 or L113, or a combination thereof, in the amino acid sequence of SEQ ID NO: 1. The gene may include a substitution of nucleotides corresponding to positions 334 to 336 in the nucleotide sequence of SEQ ID NO: 2 with TTG, nucleotides corresponding to positions 337 to 339 in the nucleotide sequence of SEQ ID NO: 2 with TTA, or a combination thereof; and a synonymous nucleic acid alteration of a codon encoding an amino acid residue corresponding to position L112 or L113, or a combination thereof, in the amino acid sequence of SEQ ID NO: 1.

The genetic modification may increase the expression of a gene that encodes a cellulose synthase. Each of the amino acid residues corresponding to positions L112, L113, L114, A115, E116, and L117 in the amino acid sequence of SEQ ID NO: 1 is an amino acid residue in a upstream region in front of a catalytic region of the cellulose synthase, expression of the cellulose synthase may increase when at least one nucleotide substitution occurs at positions 334 to 351 in the nucleotide sequence of SEQ ID NO: 2, and thus cellulose synthase activity may be increased in the cell.

The term “corresponding to” as used herein may refer to the amino acid positions of a protein of interest that aligns with the cited positions of a standard protein (positions L112, L113, L114, A115, E116, and L117 of SEQ ID NO: 1) when the protein of interest and the amino acid sequence of the standard protein (for example, SEQ ID NO: 1) are aligned using an art-acceptable protein alignment program, including the BLAST pairwise alignment, or the well-known Lipman-Pearson Protein Alignment program with the following choice of parameters: Ktuple=2, Gap Penalty=4, and Gap length penalty=12. The database in which the standard protein sequence is stored may be non-redundant proteins of NCBI. A range of “corresponding” nucleotide sequences at certain positions may be within an E-value of 0.00001 and an H-value of 0.001.

Examples of proteins having amino acid residues corresponding to positions L112, L113, L114, A115, E116, and L117 in the amino acid sequence of SEQ ID NO: 1 (hereinafter, also referred to as “homolog of cellulose synthase A”), obtained under the above-described alignment conditions, are shown in Table 1.

TABLE 1

| No. | NCBI ID |
|---|---|
| 1 | WP_048883595.1 |
| 2 | WP_053323515.1 |

The homolog may be a cellulose synthase, for example, cellulose synthase A, which originates from Komagataeibacter europaeus, Komagataeibacter hansenii, Komagataeibacter intermedius, Komagataeibacter kakiaceti, Komagataeibacter kombuchae, Komagataeibacter maltaceti, Komagataeibacter medelinensis, Komagataeibacter nataicola, Komagataeibacter oboediens, Komagataeibacter rhaeticus, Komagataeibacter saccharivorans, Komagataeibacter sucrofermentans, or Komagataeibacter swingsii.

In another embodiment, the genetic modification may include a gene encoding cellulose synthase B, diguanyl cyclase, or a combination thereof. In the recombinant microorganism including a genetic modification that increases activity of cellulose synthase A, a genetic modification that increases activity of cellulose synthase B and/or diguanyl cyclase may or may not be included.

The introduction of a gene may be implemented via a vehicle, for example, a vector. The introduced gene may be an endogenous gene or an exogenous gene. The introduced gene may be in or outside the chromosome of the microorganism. A single gene or a plurality of genes may be introduced. The number of the introduced genes may be, for example, 2 or more, 5 or more, 10 or more, 50 or more, 100 or more, or 1000 or more.

The recombinant microorganism may belong to the genus Escherichia, the genus Komagataeibacter, the genus Acetobacter, the genus Gluconobacter, or the genus Pseudomonas. For example, the recombinant microorganism may be E. coli or K. xylinus.

In another aspect of the present disclosure, a cellulose production method is provided, the method including: culturing a recombinant microorganism including a genetic modification that increases activity of a cellulose synthase, in a culture medium; and separating cellulose from the culture medium thus obtained.

In the cellulose production method, the cellulose synthase, the genetic modification, the recombinant microorganism, and the gene may be the same as described above.

The genetic modification may be introduction of a gene encoding a cellulose synthase B, a diguanyl cyclase, or a combination thereof. The cellulose synthase B and the diguanyl cyclase may also be the same as described above.

The cellulose production method may include culturing a recombinant microorganism including a genetic modification that increases activity of a cellulose synthase, in a culture medium.

The culturing may be performed in a culture medium including a carbon source, for example, glucose. The culture medium used in the culturing of the microorganism may be any general culture medium appropriate for growth of a host cell, such as a minimal medium or a complex medium including an appropriate supplement. An appropriate medium may be commercially purchased or may be prepared using a known preparation method.

The culture medium may be a medium containing selected ingredients satisfying the specific requirements of a microorganism. The culture medium may be a medium including an ingredient selected from the group consisting of a carbon source, a nitrogen source, a salt, a trace element, and a combination thereof. The culture medium may be, for example, a methyl red (MR) medium, a methyl red and voges-proskauer (MR-VP) medium, a Luria-Bertani (LB) medium, a Hestrin-Schramm (HS) medium, or a combination thereof.

A culturing condition refers to a condition for culturing the microorganism. The culturing condition may be, for example, a carbon source, a nitrogen source, or oxygen used by the microorganism. The carbon source that is usable by the microorganism may include a monosaccharide, a disaccharide, or a polysaccharide. The carbon source may be an assimilable carbon source for any microorganism. For example, the carbon source may be glucose, fructose, mannose, or galactose. The nitrogen source may be an organic nitrogen compound or an inorganic nitrogen compound. The nitrogen source may be, for example, an amino acid, an amide, an amine, a nitrate, or an ammonium salt. The oxygen condition for culturing the microorganism may be an aerobic condition at a normal partial pressure of oxygen, an atmospheric low-oxygen condition including about 0.1% to about 10% oxygen in air, or an anaerobic condition including no oxygen. A metabolic pathway of the microorganism may vary in accordance with a carbon source and a nitrogen source that are practically available.

The culturing condition may be appropriately controlled to be suitable for production of a selected product, for example, cellulose. The culturing may be performed under an aerobic or anaerobic condition for cell growth. The culturing may be static culturing without agitation. The culturing may be performed at a low concentration of the microorganism, with an OD₆₀₀ of about 3 or less. The concentration of the microorganism may be at a level which ensures that there is sufficient space so that secretion of cellulose is not inhibited.

The cellulose production method may include separating cellulose from the culture medium.

The separating may be, for example, recovering a cellulose pellicle formed on a surface of the culture medium. The cellulose pellicle may be recovered by being physically removed, or by removing the culture medium. The separating may include recovering the cellulose pellicle intact without damaging the shape of the cellulose pellicle.

In other aspects of the present disclosure, a cellulose synthase having increased activity and a polynucleotide encoding the cellulose synthase are provided. The polynucleotide may have increased expression when introduced into a cell.

The cellulose synthase may be encoded by the polynucleotide that include a synonymous nucleic acid alteration of a codon encoding an amino acid residue corresponding to position L112, L113, L114, A115, E116, or L117, or a combination thereof, in an amino acid sequence of SEQ ID NO: 1. The cellulose synthase may be encoded by the polynucleotide that include at least one nucleotide substitution of nucleotides corresponding to positions 334 to 351 in a nucleotide sequence of SEQ ID NO: 2. The polynucleotide encoding the cellulose synthase may have a synonymous nucleic acid alteration of a codon encoding an amino acid residue corresponding to position L112, L113, L114, A115, E116, or L117, or a combination thereof, in an amino acid sequence of SEQ ID NO: 1. The polynucleotide encoding the cellulose synthase may have a synonymous nucleic acid alteration of a codon encoding an amino acid residue corresponding to position L112, L113, or a combination thereof, in the amino acid sequence of SEQ ID NO: 1.

The synonymous nucleic acid alteration may include at least one nucleotide substitution of nucleotides corresponding to positions 334 to 351 in a nucleotide sequence of SEQ ID NO: 2 that encode a polypeptide including the amino acid sequence of SEQ ID NO: 1. The synonymous nucleic acid alteration may include at least one nucleotide substitution of nucleotides corresponding to positions 334 to 339 in the nucleotide sequence of SEQ ID NO: 2. The synonymous nucleic acid alteration may include a substitution of nucleotides corresponding to positions 334 to 336 in the nucleotide sequence of SEQ ID NO: 2 with TTG, a substitution of nucleotides corresponding to positions 337 to 339 in the nucleotide sequence of SEQ ID NO: 2 with TTA, or a combination thereof.

The polynucleotide may be included in a vector. The vector may be any vector that may be used to introduce a polynucleotide into a microorganism. The vector may be, for example, a plasmid or a viral vector.

In another aspect of the present disclosure, a method of constructing a recombinant microorganism including a genetic modification that increases activity of a cellulose synthase is provided. The recombinant microorganism may have improved cellulose productivity. The method may include introducing a gene encoding a cellulose synthase having increased activity, such as any of the polynucleotide or gene described herein, into a microorganism. The introducing of the gene encoding a cellulose synthase having increased activity may include introducing a vehicle into the microorganism, the vehicle including the gene encoding a cellulose synthase having increased activity. The method may include introducing a genetic modification that increases activity of a cellulose synthase B and/or a diguanyl cyclase into the microorganism.

In the method of constructing a recombinant microorganism, the cellulose synthase, the genetic modification, the microorganism, and the gene may be the same as described above.

In an aspect of the present disclosure, a recombinant microorganism including a genetic modification that increases activity of a cellulose synthase, according to any of the above-described embodiments, may be used to produce cellulose with high efficiency.

In another aspect of the present disclosure, by a method of producing cellulose, according to any of the above-described embodiments, cellulose may be efficiently produced.

In other aspects of the present disclosure, a cellulose synthase having increased activity, and a polynucleotide encoding the cellulose synthase, according to any of the above-described embodiments, may be used to produce cellulose with high efficiency.

In another aspect of the present disclosure, by a method of constructing a recombinant microorganism including a genetic modification that increases activity of a cellulose synthase, according to any of the above-described embodiments, the recombinant microorganism having increased cellulose productivity may be efficiently constructed.

One or more embodiments of the present disclosure will now be described in detail with reference to the following examples. However, these examples are only for illustrative purposes and are not intended to limit the scope of the one or more embodiments of the present disclosure.

### Example 1. Construction of E. coli Including Cellulose Synthase A (bcsA), and Production of Cellulose

In the present example, a nucleotide sequence that regulates the expression of a gene encoding a cellulose synthase A was screened. The gene was introduced into E. coli (DE3) (Novagene 69450), the gene encoding the cellulose synthase A having increased activity. The microorganism into which the gene was introduced was cultured to produce cellulose. An effect of the nucleotide sequence in the gene on cellulose productivity, wherein the nucleotide sequence regulates the expression of the cellulose synthase A, was investigated.

Confirmation 1: Expression Level of Truncated Fragment of Cellulose Synthase A Gene, and Nucleotide Sequence that Regulates Expression of the Gene

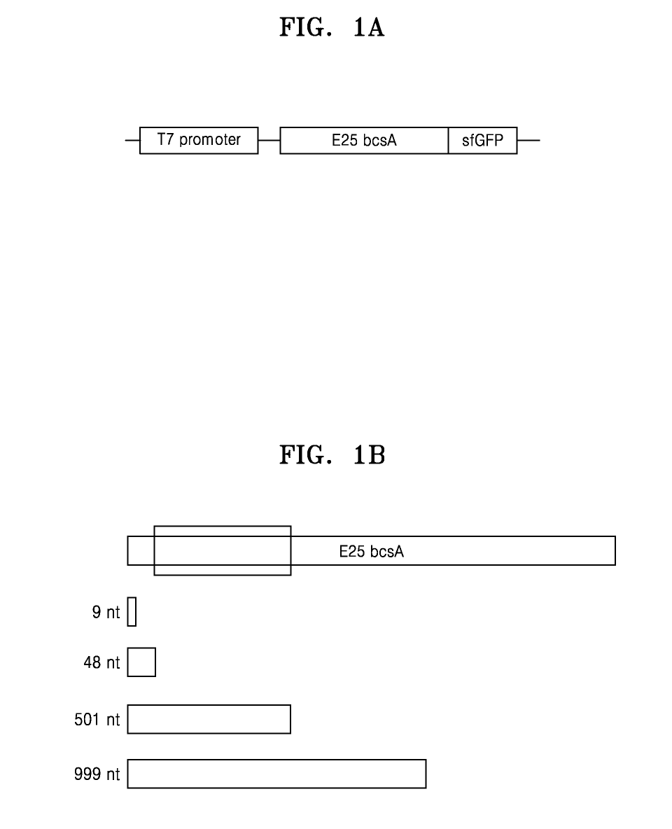

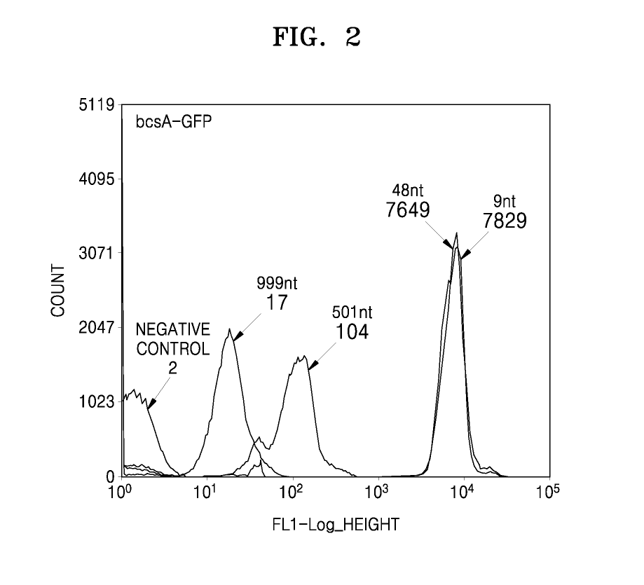

The cellulose synthase A gene was amplified by polymerization chain reaction (PCR) using the genome of K. xylinus E25 as a template and SEQ ID NOs: 7 and 8 as primers. Cellulose synthase A gene that is codon-optimized for E. coli was also synthesized and constructed, based on the cellulose synthase gene of K. xylinus E25 (General Biosystems, Inc.). The cellulose synthase A gene having a total length of 2238nt was truncated stepwise. The cellulose synthase A gene included a nucleotide sequence of SEQ ID NO: 2, and its codon-optimized sequence included a nucleotide sequence of SEQ ID NO: 6. The four truncated fragments had a size of 9nt, 48nt, 501nt, and 999nt, respectively. FIG. 1B illustrates structures of these four fragments. The four fragments were named bcsA (E25, 9nt), bcsA (E25, 48nt), bcsA (E25, 501nt), and bcsA (E25, 999nt), respectively.

TABLE 2

| | | 5′-3′ |
|---|---|---|
| SEQ ID NO: 7 | Forward | AAGGCCTTCATATGATGTCAGAGGTTCAG |
| (primer 7) | | TCGTCAG |
| SEQ ID NO: 8 | Backward | AAGGCCTTGCGGCCGCTTACGAGGCCGCA |
| (primer 8) | | CGACTGA |

A gene that encodes a green fluorescent protein (GFP) was constructed as follows. After synthesis of a DNA sequence for superfold GFP (sfGFP) (General Biosystems), PCR was performed with SEQ ID NOs: 9 and 10 as primers to amplify the sfGFP gene. This sfGFP gene serves as a reporter of promoter activity.

TABLE 3

| | | 5′-3′ |
|---|---|---|
| SEQ ID NO: 9 | forward | AAGGCCTTGCGGCCGCATGAGCAAAGGA |
| (primer 9) | | GAAGAACTTTTCAC |
| SEQ ID NO: 10 | backward | AAGGCCTTGCGGCCGCTTATTTGTAGAG |
| (primer 10) | | CTCATCCATGCCAT |

Cloning was performed by cleaving the four fragments of the cellulose synthase A gene and a pET22b vector having a T7 promoter with restriction enzymes NdeI and NotI, and then performing ligation using a T4 DNA ligase, to obtain expression vectors pET22b-bcsA (E25, 9nt), pET22b-bcsA (E25, 48nt), pET22b-bcsA (E25, 501nt), and pET22b-bcsA (E25, 999nt) including the respective truncated fragments of the cellulose synthase A gene.

Subsequently, cloning was performed by cleaving the expression vectors pET22b-bcsA (E25, 9nt), pET22b-bcsA (E25, 48nt), pET22b-bcsA (E25, 501nt), and pET22b-bcsA (E25, 999nt), and the amplified sfGFP, with restriction enzyme NotI, and then performing ligation using a T4 DNA ligase to obtain expression vectors pET22b-bcsA (E25, 9nt)-GFP, pET22b-bcsA (E25,48nt)-GFP, pET22b-bcsA (E25, 501nt)-GFP, and pET22b-bcsA (E25, 999nt)-GFP including the respective truncated fragments of the cellulose synthase A gene and the sfGFP.

FIG. 1A illustrates the structure of an expression vector including the T7 promoter, a cellulose synthase A gene fragment, and a sfGPF-tag. These vectors including the respective four fragments of the cellulose synthase A gene were named pET22b-bcsA (E25, 9nt)-GFP, pET22b-bcsA (E25,48nt)-GFP, pET22b-bcsA (E25, 501nt)-GFP, and pET22b-bcsA (E25, 999nt)-GFP, respectively.

The pET22b-bcsA (E25, 9nt)-GFP, pET22b-bcsA (E25,48nt)-GFP, pET22b-bcsA (E25, 501nt)-GFP, and pET22b-bcsA (E25, 999nt)-GFP vectors were transformed into E. coli BL21(DE3) codon RP plus by heat shock. The transformed E. coli were inoculated into a 1 L-flask containing about 200 mL of each of an MR medium and an LB medium and cultured at about 30° C. for about 24 hours while stirring at about 250 rpm. The cultured strain was recovered by centrifugation at about 6,000 rpm for about 5 minutes, washed, and then suspended in a phosphate buffered saline (PBS).

Fluorescent cells including the expressed GFP were screened by flow cytometry, and quantified using a Moflo XDP flow cytometer (Beckman Coulter, Brea, Calif., USA). Cells were excited with blue light having a wavelength of about 488 nm by using an air-cooled argon ion laser. The fluorescent GFP signals of the cells were detected with an FL1 (530/40 nm) channel. An average fluorescence level was recorded using MoFlo™ XDP SUMMIT software version 5.2 (Beckman Coulter).

When a 501nt or 999nt fragment of the cellulose synthase A gene was expressed, the expressed amount of the reporter protein GFP was remarkably reduced as compared to when a 9nt or 48nt fragment of the cellulose synthase A gene was expressed.

This indicates that a nucleotide sequence at a site between 48nt and 501nt of the cellulose synthase A gene regulates or inhibits the expression of the cellulose synthase A gene.

(2) Confirmation 2: Expression Level of Truncated Fragment of Cellulose Synthase A Gene, and Nucleotide Sequence that Regulates Expression of the Gene

The nucleotides between 334nt and 351nt of the cellulose synthase A gene were selected from the nucleotides between 48nt to 501nt of the cellulose synthase A gene to identify whether the nucleotides between 334nt and 351nt regulate or inhibit expression of the cellulose synthase A gene.

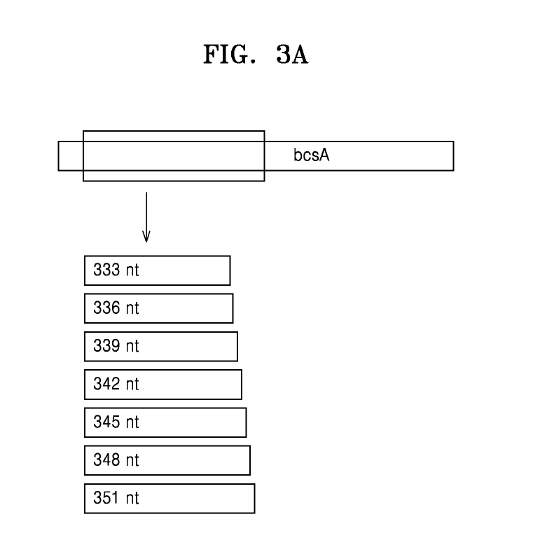

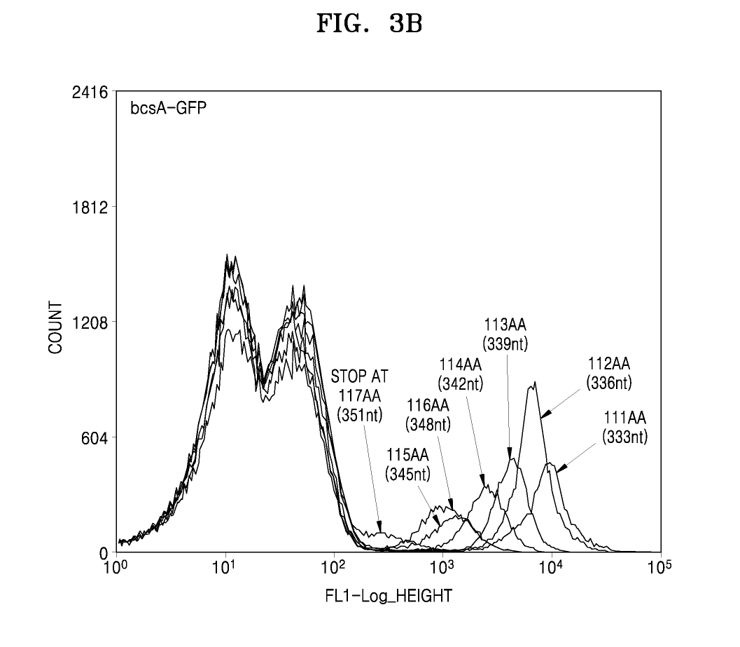

The nucleotides between 334nt and 351nt of the cellulose synthase A gene were truncated into 3nt units, each encoding an amino acid. FIG. 3A illustrates seven fragments of the cellulose synthase A gene, that is, 333nt, 336nt, 339nt, 342nt, 345nt, 348nt, and 351nt. These seven gene fragments were named bcsA (E25, 333nt), bcsA (E25, 336nt), bcsA (E25, 339nt), bcsA (E25, 342nt), bcsA (E25, 345nt), bcsA (E25, 348nt), and bcsA (E25, 351nt), respectively.

Expression vectors including GFP and the respective seven fragments of the cellulose synthase A gene were obtained in the same manner as described above in Section (1). The vectors were named pET22b-bcsA (E25, 333nt)-GFP, pET22b-bcsA (E25, 336nt)-GFP, pET22b-bcsA (E25, 339nt)-GFP, pET22b-bcsA (E25, 342nt)-GFP, pET22b-bcsA (E25, 345nt)-GFP, pET22b-bcsA (E25, 348nt)-GFP, and pET22b-bcsA (E25, 351nt)-GFP.

Each of the vectors was transformed into E. coli BL21(DE3) codon RP plus and cultured in the same manner as described above in Section (1). Fluorescent cells including expressed GFP were screened using a flow cytometer in the same manner as described above in Section (1).

FIG. 3B illustrates results of counting cells including expressed GFP after the transformation of the vectors pET22b-bcsA (E25, 333nt)-GFP, pET22b-bcsA (E25, 336nt)-GFP, pET22b-bcsA (E25, 339nt)-GFP, pET22b-bcsA (E25, 342nt)-GFP, pET22b-bcsA (E25, 345nt)-GFP, pET22b-bcsA (E25, 348nt)-GFP, and pET22b-bcsA (E25, 351nt)-GFP into the cells.

Referring to FIG. 3B, as the number of amino acids increased beyond the 111th amino acid, the expressed amount of the reporter protein GFP gradually reduced. This indicates that the nucleotides between 334nt and 355nt of the cellulose synthase A gene include a nucleotide sequence that regulates or inhibits the expression of the cellulose synthase A gene.

(3) Conformation of Expression Increase by Nucleotide Substitution in Nucleotide Sequence that Regulates Expression

It was identified whether the expression of the cellulose synthase A gene encoding a cellulose synthase increased by a nucleotide substitution between positions 334 and 351 of a nucleotide sequence thereof.

In particular, in the 336nt cellulose synthase A gene fragment, nucleotides corresponding to positions 334 to 336 of a nucleotide sequence of SEQ ID NO: 2, that is, CTG, were substituted with TTG. The resulting fragment was named bcsA (E25, 336nt, 334-336 mod).

In the 339nt cellulose synthase A gene fragment, nucleotides corresponding to positions 334 to 336 of the nucleotide sequence of SEQ ID NO: 2, that is, CTG, were substituted with TTG, and nucleotides corresponding to positions 337 to 339 of the nucleotide sequence of SEQ ID NO: 2, that is, CTG, were substituted with TTA. The resulting fragment was named bcsA (E25, 339nt, 334-336, 337-339 mod).

In the 351nt cellulose synthase A gene fragment, nucleotides corresponding to positions 334 to 336 of the nucleotide sequence of SEQ ID NO: 2, that is, CTG, were substituted with TTG, and nucleotides corresponding to positions 337 to 339 of the nucleotide sequence of SEQ ID NO: 2, that is, CTG, were substituted with TTA. The resulting fragment was named bcsA (E25, 351nt, 334-336, 337-339 mod).

No modification in amino acids resulted from the nucleotide substitutions.

Expression vectors including GPF and the respective cellulose synthase A gene fragments in which the nucleotides corresponding to positions 334 to 336 and/or the nucleotides corresponding to positions 337 to 339 of the nucleotide sequence of SEQ ID NO: 2 were substituted were obtained in the same manner as described above in Section (1). The vectors were named pET22b-bcsA (E25, 336nt, 334-336 mod)-GFP, pET22b-bcsA (E25, 339nt, 334-336, 337-339 mod)-GFP, and pET22b-bcsA (E25, 351nt, 334-336, 337-339 mod)-GFP, respectively.

Each of the vectors was transformed into E. coli BL21(DE3) codon RP plus and cultured in the same manner as described above in Section (1). Fluorescent cells including expressed GFP were screened using a flow cytometer in the same manner as described above in Section (1).

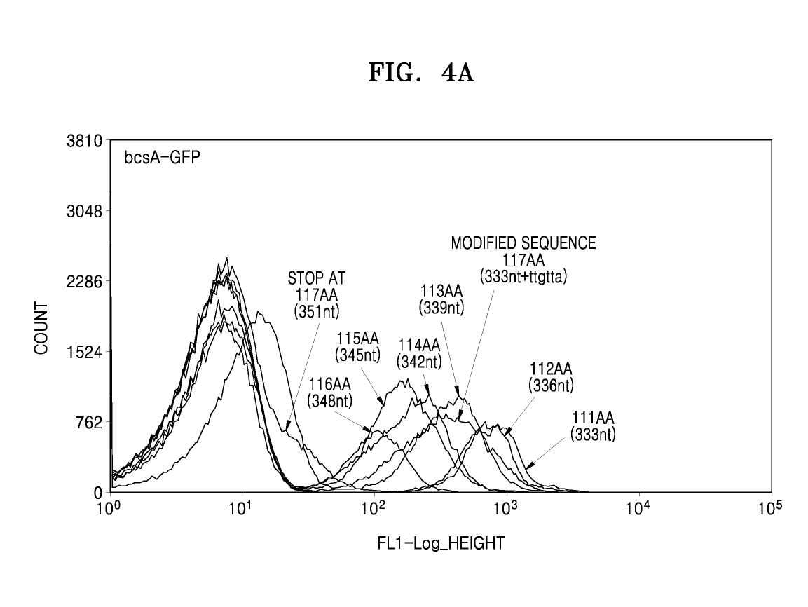

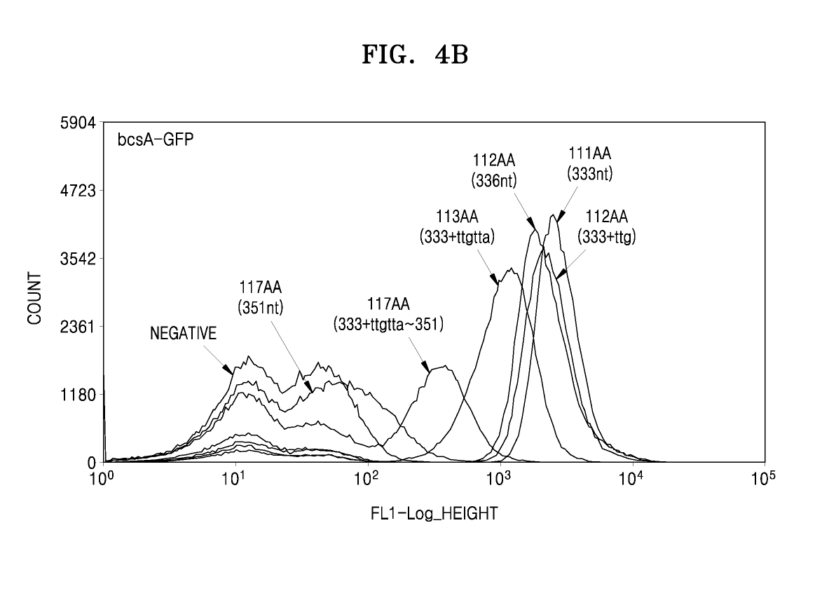

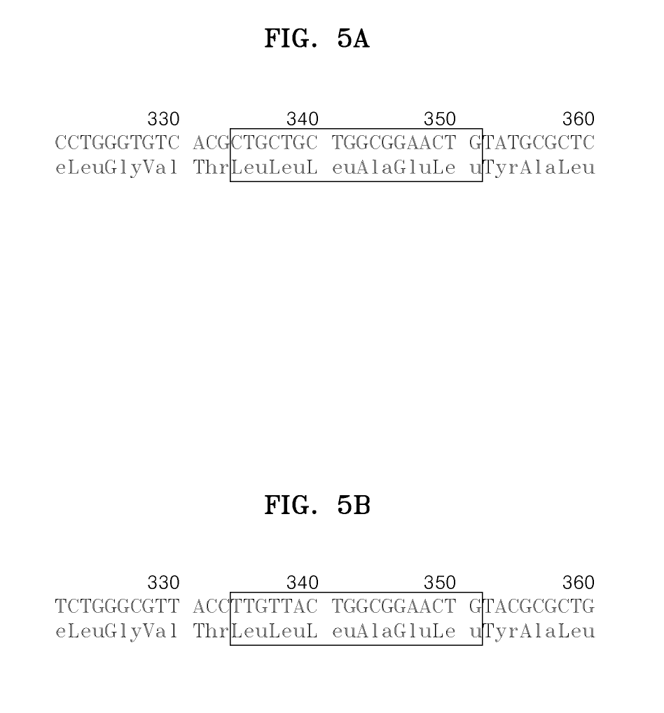

FIG. 5A illustrates a nucleotide sequence that regulates the expression of a cellulose synthase A gene of K. xylinus E25. FIG. 4A illustrates the results of counting cells including expressed GFP after the transformation of the vectors pET22b-bcsA (E25, 333nt)-GFP, pET22b-bcsA (E25, 336nt)-GFP, pET22b-bcsA (E25, 339nt)-GFP, pET22b-bcsA (E25, 342nt)-GFP, pET22b-bcsA (E25, 345nt)-GFP, pET22b-bcsA (E25, 348nt)-GFP, pET22b-bcsA (E25, 351nt)-GFP, and pET22b-bcsA (E25, 351nt, 334-336, 337-339 mod)-GFP into the cells. Referring to FIG. 4A, as the number of amino acids increases beyond the 111^{th} amino acid, the expressed amount of the reporter protein GFP gradually reduced. In the cells including the substituted nucleotides corresponding to positions 334 to 336 and positions 337 to 339 of the nucleotide sequence of SEQ ID NO: 2 and including a 117^{th} amino acid, the expressed amount of the reporter protein GFP increased remarkably as compared with the cells including a 117^{th} amino acid but having no nucleotide substitutions.

FIG. 4B illustrates the results of counting cells including expressed GFP after transformation of expression vectors including fragments of the cellulose synthase A gene originating from K. xylinus E25 into the cells, the expression vectors named pET22b-bcsA (E25, 333nt)-GFP, pET22b-bcsA (E25, 336nt)-GFP, pET22b-bcsA (E25, 336nt, 334-336 mod)-GFP, pET22b-bcsA (E25, 339nt, 334-336, 337-339 mod)-GFP, pET22b-bcsA (E25, 351nt)-GFP, and pET22b-bcsA (E25, 351nt, 334-336, 337-339 mod)-GFP, respectively. Referring to FIG. 4B, in the cells including the nucleotides TTG, substituted in place of CTG, at positions 334 to 336 of the nucleotide sequence of SEQ ID NO: 2, and also including the 112^{th} amino acid, the expressed amount of the reporter protein GFP was increased as compared with the cells including the 112^{th} amino acid but without any nucleotide substitution.

In comparison of FIG. 4A and FIG. 4B, in the cases including the 112^{th} amino acid and the 113^{th} amino acid with the substituted nucleotides corresponding to positions 334 to 336 of the nucleotide sequence of SEQ ID NO: 2, the expressed amounts of the reporter protein GFP were increased as compared with the cases of including the 112^{th} amino acid and the 113^{th} amino acid but without any nucleotide substitution, respectively.

These results indicate that the nucleotides between 334nt and 336nt and/or the nucleotides between 337nt and 339nt in the cellulose synthase A gene include a nucleotide sequence that regulates or inhibits the expression of the cellulose synthase A gene.

(4) Confirmation of the Expression Level of Truncated Fragment of Cellulose Synthase A Gene from a Different Strain of the Genus Komagataeibacter, and the Nucleotide Sequence that Regulates Expression of the Gene

The cellulose synthase A gene was amplified by performing PCR using the genome of K. xylinus KCCM 41431 as a template and nucleotide sequences of SEQ ID NOs: 11 and 12 as primers. The cellulose synthase A gene of the K. xylinus KCCM 41431 included a nucleotide sequence of SEQ ID NO: 15. The nucleotides corresponding to positions between 334nt and 351nt of the cellulose synthase A gene having a total length of 2265nt were screened to identify whether the nucleotides include a nucleotide sequence that regulates or inhibits expression of the cellulose synthase A gene.

TABLE 4

| | | 5′-3′ |
|---|---|---|
| SEQ ID NO: 11 | forward | AAGGCCTTTCTAGAAATAATTTTGTTTAA |
| (primer 11) | | CTTTAAGAAGGAGATATAATGTCAGAGGT |
| | | TCAGTCGCC |
| SEQ ID NO: 12 | backward | AAGGCCTTGCGGCCGCTCACGAGGCCGCA |
| (primer 12) | | CGGCT |

The nucleotides between 334nt and 351nt of the cellulose synthase A gene of K. xylinus KCCM 41431 were truncated into 3 nt units each encoding an amino acid, in the same manner as in Section (1), except that restriction enzymes XbaI and NotI were used, to thereby construct fragments of the cellulose synthase A gene. The seven fragments of the cellulose synthase A gene were of 333nt, 336nt, 339nt, 342nt, 345nt, 348nt, and 351nt, and were respectively named bcsA (41431, 333nt), bcsA (41431,336nt), bcsA (41431, 339nt), bcsA (41431, 342nt), bcsA (41431, 345nt), bcsA (41431, 348nt), and bcsA (41431, 351nt).

Expression vectors including GFP and the respective seven fragments of the cellulose synthase A gene were obtained in the same manner as described above in Section (1). The vectors were named pET22b-bcsA (41431, 333nt)-GFP, pET22b-bcsA (41431, 336nt)-GFP, pET22b-bcsA (41431, 339nt)-GFP, pET22b-bcsA (41431, 342nt)-GFP, pET22b-bcsA (41431, 345nt)-GFP, pET22b-bcsA (41431, 348nt)-GFP, and pET22b-bcsA (41431, 351nt)-GFP, respectively.

Each of the vectors was transformed into E. coli BL21(DE3) codon RP plus and cultured in the same manner as described above in Section (1). Fluorescent cells including expressed GFP were screened using a flow cytometer in the same manner as described above in Section (1).

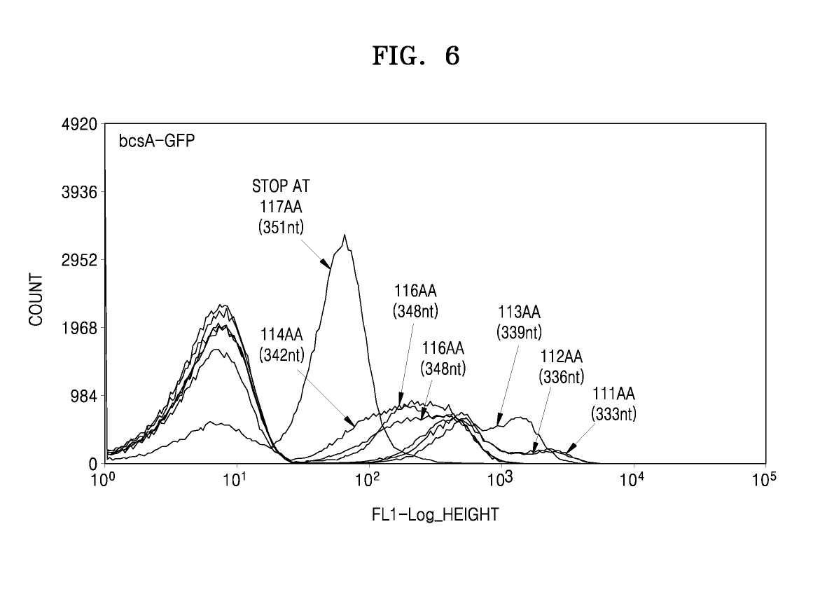

FIG. 5B illustrates a nucleotide sequence that encodes a cellulose synthase A having increased activity. FIG. 6 illustrates the results of counting cells including expressed GFP after the transformation of the expression vectors including the fragments of the cellulose synthase A gene originating from K. xylinus KCCM 41431 into the cells, the expression vectors respectively named pET22b-bcsA (41431, 333nt)-GFP, pET22b-bcsA (41431, 336nt)-GFP, pET22b-bcsA (41431, 339nt)-GFP, pET22b-bcsA (41431, 342nt)-GFP, pET22b-bcsA (41431, 345nt)-GFP, pET22b-bcsA (41431, 348nt)-GFP, and pET22b-bcsA (41431, 351nt)-GFP. Referring to FIG. 6, as the number of amino acids increased beyond the 111^{th} amino acid, the expressed amount of the reporter protein GFP gradually reduced. This indicates that the nucleotides between 334nt and 351nt are still important in a different strain of the genus of Komagataeibacter for the regulation or inhibition of expression of the cellulose synthase A gene.

(5) Construction of E. coli Including Introduced Gene that Encodes Cellulose Synthase Having Increased Activity, and Identification of Cellulose Yield

A cellulose synthase gene having increased activity was introduced into E. coli. Detailed gene introduction processes are as follows.

A nucleotide sequence of a cellulose synthase A gene (SEQ ID NO: 6) of K. xylinus E25 that is codon-optimized for E. coli and a nucleotide sequence of a cellulose synthase A gene having increased activity (SEQ ID NO: 4) were cloned into a pET28a vector (Novagen, #69864) at NdeI and NotI restriction enzyme sites with an In-fusion HD cloning kit (#PT5162-1, Clontech), thereby obtaining cellulose synthase expression vectors pET-bcsA (E25, 2238nt) and pET-bcsA (E25, 2238nt, 334-336, 337-339 mod).

A nucleotide sequence of a cellulose synthase B gene (SEQ ID NO: 13) of K. xylinus E25 was cloned into the pET-bcsA(E25, 2238nt) and pET-bcsA(E25, 2238nt, 334-336, 337-339 mod) vectors at the NotI restriction enzyme site thereof with the In-fusion HD cloning kit, thereby obtaining cellulose synthase expression vectors pET-bcsA(E25, 2238nt)B and pET-bcsA(E25, 2238nt, 334-336, 337-339 mod)B, respectively.

A nucleotide sequence of a diguanyl cyclase gene (SEQ ID NO: 14) of Thermotoga maritima MSB8 (GenBank Accession Number: NC_000853) was cloned into a pACYC-duet (Novagen, #71147) vector at EcoRI and HindIII restriction enzymes sites with the In-fusion HD cloning kit, to thereby obtain the diguanyl cyclase gene expression vector pACYC-DGC.

The obtained expression vectors were transformed into E. coli BL21(DE3) codon RP plus by heat shock.

Whether the expression vectors were introduced or not was identified by sequencing the transformed E. coli BL21(DE3) codon RP plus strain. The transformed E. coli BL21(DE3) codon RP plus strain was smeared on LB and MR media each including 50 μg/ml of ampicillin and 25 μg/ml of kanamycin, and then cultured at about 30° C. Then, strains having resistance against ampicillin and kanamycin were screened, thereby identifying whether the expression vectors were introduced or not.

The LB medium used contained 10 g/L of tryptone, 5 g/L of yeast extract, and 5 g/L of NaCl. The MR medium used contained 6.67 g of KH₂PO₄, 4 g of (NH₄)₂HPO₄, 0.8 g of MgSO₄.7H₂O, and 0.8 g of citric acid. The LB and MR media both contained 5 ml of a trace metal solution. The trace metal solution (per liter) included 10 g of FeSO₄.7H₂O, 2 g of CaCl₂, 2.2 g of ZnSO₄.7H₂O, 0.5 g of MnSO₄.4H₂O, 1 g of CuSO₄.5H₂O, 0.1 g of (NH₄)₆Mo₇O₂₄.4H₂O, and 0.02 g of Na₂B₄O₇.10H₂O. To each of the LB and MR media, glucose and thiamine were added to reach a final concentration of 20 g/L (glucose) and 10 mg/L (thiamine), respectively, in each of the media.

The E. coli BL21(DE3) codon RP plus including the pET-bcsA(E25, 2238nt)B and pACYC-DGC vectors introduced thereinto, and the E. coli BL21(DE3) codon RP plus including the pET-bcsA(E25, 2238nt, 334-336, 337-339 mod)B and pACYC-DGC vectors introduced thereinto were named E. coli RP/pET-bcsA(E25, 2238nt)B+pACYC-DGC, and E. coli RP/pET-bcsA(E25, 2238nt, 334-336, 337-339 mod)B+pACYC-DGC, respectively.

E. coli RP/pET-bcsA(E25, 2238nt)B+pACYC-DGC, E. coli RP/pET-bcsA(E25, 2238nt, 334-336, 337-339 mod)B+pACYC-DGC, and E. coli RP strains were respectively inoculated in 1 L-flasks each containing about 200 mL of the MR medium, and then 0.4 mM of isopropyl-β-thiogalactoside (IPTG) was added into each of the flasks to induce production of protein from the genes at an OD₆₀₀ of 0.5. Then, incubation was performed at about 30° C. for about 4 hours, and then for a further about 24 hours while stirring at about 250 rpm. Each strain was recovered, washed, and then suspended in about 5 mL of KH₂PO₄. After 2 mL portions were separated from the total 5 mL of the suspension, 700 units/g of cellulase were added to a 2 mL portion of the suspension, and no cellulase was added to the other 2 mL portion of the suspension. The two suspensions were incubated in static conditions at about 50° C. for about 24 hours, and then the concentration of glucose as a product resulting from the degradation of cellulose in each of the suspensions was measured. The glucose concentration measurement was performed using a high-performance liquid chromatography (HPLC) system equipped with an Aminex HPX-87H column (available from Bio-Rad, USA).

In a similar manner as above, E. coli RP/pET-bcsA(E25, 2238nt)B+pACYC-DGC, E. coli RP/pET-bcsA(E25, 2238nt, 334-336, 337-339 mod)B+pACYC-DGC, and E. coli RP strains were incubated in a Luria-Bertani (LB) medium, and then incubated with cellulase to measure the concentration of the resulting cellulose degradation product.

As a result of the 4-hour incubation in the MR and LB media (not shown), a trace of cellulose nanofiber (CNF) was detected in the E. coli RP/pET-bcsA (E25, 2238nt, 334-336, 337-339 mod)B+pACYC-DGC strain.

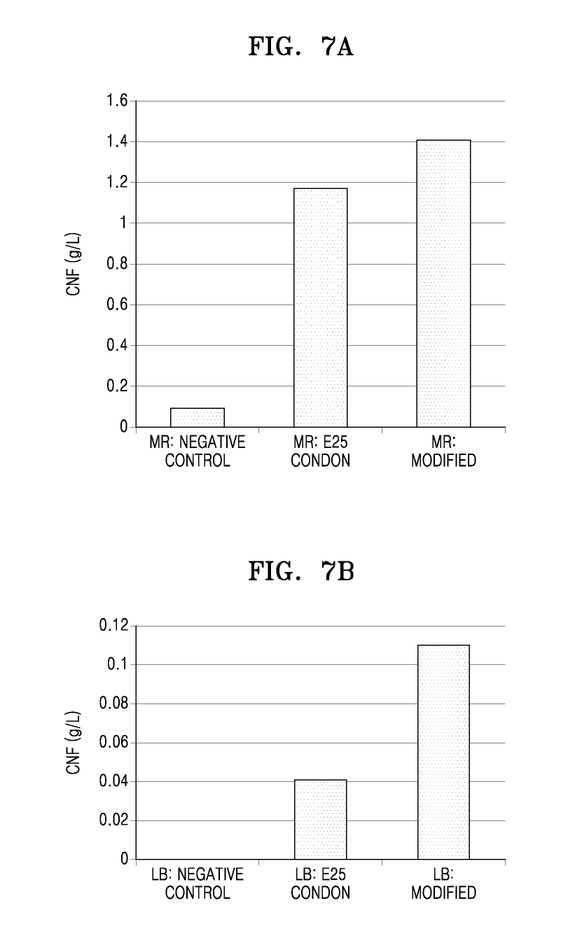

The results of the 24-hour incubation are shown in FIGS. 7A and 7B. FIG. 7A illustrates the yields of CNF after the 24-hour incubation of E. coli RP/pET-bcsA(E25, 2238nt)B+pACYC-DGC, E. coli RP/pET-bcsA(E25, 2238nt, 334-336, 337-339 mod)B+pACYC-DGC, and E. coli RP strains in the MR medium. Referring to FIG. 7A, when the E. coli including the introduced cellulose synthase gene having increased activity was incubated in the MR medium, a yield of CNF increased by about 29% relative to that of the control group. In another experimental group in which the E. coli including the introduced cellulose synthase gene having increased activity was incubated in the MR medium, a yield of CNF was about 1.49 g/L (not shown).

FIG. 7B illustrates the yields of CNF after the 24-hour incubation of E. coli RP/pET-bcsA(E25, 2238nt)B+pACYC-DGC, E. coli RP/pET-bcsA(E25, 2238nt, 334-336, 337-339 mod)B+pACYC-DGC, and E. coli RP strains in the LB media. Referring to FIG. 7B, when the E. coli including the introduced cellulose synthase gene having increased activity was incubated in the LB medium, a yield of CNF improved by about 168% relative to that of the control group.

TABLE 5

| | Strain | | |
|---|---|---|---|
| | | E. coli RP/pET- | |
| | | bcsA(E25, | E. coli RP/pET-bcsA(E25, |
| Product of | E. coli | 2238 nt)B + pACYC- | 2238 nt, 334-336, 337-339 |
| culture | RP | DGC | mod)B + pACYC-DGC |
| CNF (g/L) | 0 | 0.041 | 0.11 |

The cellulose synthase having increased activity affected the production of cellulose in the microorganism. This result indicates that nucleotides corresponding to positions 334nt to 351nt of the nucleotide sequence of the cellulose synthase A gene regulate or inhibit the expression of cellulose synthase A.

### Example 2. Construction of K. xylinus Including Cellulose Synthase A (bcsA) and Production of Cellulose by Using the K. xylinus

A bcsA (E25, 2238nt) or bcsA (E25, 2238nt, 334-336, 337-339 mod) fragment, a gapA promoter region (SEQ ID NO: 16) of K. xylinus KCCM, and a pBBR122 vector (MoBiTec) were cleaved with a PstI restriction enzyme, and then cloned using an In-fusion HD cloning kit, thereby obtaining cellulose synthase expression vectors pBBR-bcsA (E25, 2238nt) and pBBR-bcsA (E25, 2238nt, 334-336, 337-339 mod). The gapA promoter was amplified using a set of primers 3 and 5.

The constructed vectors were each introduced into K. xylinus KCCM 41431 by electroporation, and each resulting strain was inoculated on a Hestrin-Schramm (HS) agar medium including 100 mg/ml of chloramphenicol so as to yield colonies. The K. xylinus KCCM 41431 strains, respectively including the pBBR-bcsA (E25, 2238nt) and pBBR-bcsA (E25, 2238nt, 334-336, 337-339 mod) vectors which were introduced thereinto, were respectively named K. xylinus KCCM 41431/pBBR-bcsA(E25, 2238nt) and K. xylinus KCCM 41431/pBBR-bcsA(E25, 2238nt, 334-336, 337-339 mod). The HS agar medium used contained 2.0% of glucose, 0.5% of peptone, 0.5% of yeast extract, 0.27% of disodium phosphate, 0.115% of citric acid, and 1.5% of agar. The obtained colonies were inoculated in 125-mL flasks, each containing 25 mL of an HS medium, and cultured at about 30° C. while being stirred at about 250 rpm for 5 days. The HS medium used contained 2.0% of glucose, 0.5% of peptone, 0.5% of yeast extract, 0.27% of disodium phosphate, and 0.115% of citric acid. The resulting solid cellulose in each flask was washed with a 0.1N sodium hydroxide solution and water, dried in a 60° C. oven, and a yield of cellulose thus obtained was measured by weighing.

When K. xylinus including the cellulose synthase gene having increased activity introduced thereinto was incubated in the HS medium, the yield of CNF was about 1.04 g/L, which is an increase of about 12% relative to that of the control group.

TABLE 6

| | | 5′-3′ |
|---|---|---|
| SEQ ID NO: 3 | forward | CTGCCCCCCGAGACCAACTTCGGCGGCG |
| (primer 3) | | CCCGAGCGTGA |
| SEQ ID NO: 5 | backward | AAGGTCTGACATTTCACACACTGACATC |
| (primer 5) | | GGCCG |

TABLE 7

| | Strain | |
|---|---|---|
| | | K. xylinus |
| | K. xylinus | KCCM 41431/pBBR- |
| | KCCM41431/pBBR- | bcsA(E25, 2238 nt, 334-336, |
| Product of culture | bcsA(E25, 2238 nt) | 337-339 mod) |
| CNF (g/L) | 0.93 | 1.04 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising a genetic modification that increases activity of a cellulose synthase.

2. The recombinant microorganism of claim 1, wherein the genetic modification comprises an exogenous gene encoding a cellulose synthase having an activity belonging to EC 2.4.1.12, the gene having at least one nucleotide substitution of nucleotides corresponding to positions 334 to 351 in a nucleotide sequence of SEQ ID NO: 2.

3. The recombinant microorganism of claim 2, wherein the gene comprises at least one nucleotide substitution of nucleotides corresponding to positions 334 to 339 in the nucleotide sequence of SEQ ID NO: 2.

4. The recombinant microorganism of claim 2, wherein the gene encoding a cellulose synthase comprises a substitution of nucleotides corresponding to positions 334 to 336 in the nucleotide sequence of SEQ ID NO: 2 with TTG, TTA, CTT, CTC, or CTA; a substitution of nucleotides corresponding to positions 337 to 339 in the nucleotide sequence of SEQ ID NO: 2 with TTG, TTA, CTT, CTC, or CTA; or a combination thereof.

5. The recombinant microorganism of claim 2, wherein the gene encoding cellulose synthase comprises a synonymous nucleic acid alteration of a codon encoding an amino acid residue corresponding to position L112, L113, L114, A115, E116, or L117, or a combination thereof, in an amino acid sequence of SEQ ID NO: 1.

6. The recombinant microorganism of claim 3, wherein the gene encoding cellulose synthase comprises a synonymous nucleic acid alteration of a nucleotide encoding an amino acid residue corresponding to position L112, L113, or a combination thereof, in an amino acid sequence of SEQ ID NO: 1.

7. The recombinant microorganism of claim 1, wherein the genetic modification further comprises an exogenous gene encoding a cellulose synthase B, a diguanyl cyclase, or a combination thereof.

8. The recombinant microorganism of claim 1, wherein the recombinant microorganism belongs to the genus Escherichia, the genus Komagataeibacter, the genus Acetobacter, the genus Gluconobacter, or the genus Pseudomonas.

9. A method of producing cellulose, the method comprising:
culturing a recombinant microorganism having a genetic modification that increases activity of a cellulose synthase in a culture medium; and
separating cellulose from the culture medium.

10. The method of claim 9, wherein the recombinant microorganism comprises an exogenous gene encoding a cellulose synthase having an activity belonging to EC 2.4.1.12, the gene having at least one nucleotide substitution of nucleotides corresponding to positions 334 to 351 in a nucleotide sequence of SEQ ID NO: 2.

11. The method of claim 10, wherein the gene comprises at least one nucleotide substitution of nucleotides corresponding to positions 334 to 339 in the nucleotide sequence of SEQ ID NO: 2.

12. The method of claim 10, wherein the gene encoding a cellulose synthase comprises a substitution of nucleotides corresponding to positions 334 to 336 in the nucleotide sequence of SEQ ID NO: 2 with TTG, TTA, CTT, CTC, or CTA; a substitution of nucleotides corresponding to positions 337 to 339 in the nucleotide sequence of SEQ ID NO: 2 with TTG, TTA, CTT, CTC, or CTA; or a combination thereof.

13. The method of claim 10, wherein the gene encoding cellulose synthase comprises a synonymous nucleic acid alteration of a codon encoding an amino acid residue corresponding to position L112, L113, L114, A115, E116, or L117, or a combination thereof, in an amino acid sequence of SEQ ID NO: 1.

14. The method of claim 9, wherein the genetic modification further comprises an exogenous gene encoding a cellulose synthase B, a diguanyl cyclase, or a combination thereof.

15. The method of claim 9, wherein the recombinant microorganism belongs to the genus Escherichia, the genus Komagataeibacter, the genus Acetobacter, the genus Gluconobacter, or the genus Pseudomonas.

16. The method of claim 9, wherein the culture medium is a methyl red (MR) medium, a Luria-Bertani (LB) medium, a Hestrin-Schramm (HS) medium, or a combination thereof.

17. A polynucleotide encoding cellulose synthase having an activity belonging to EC 2.4.1.12, the polynucleotide having at least one nucleotide substitution of nucleotides corresponding to positions 334 to 351 in a nucleotide sequence of SEQ ID NO: 2.

18. The polynucleotide of claim 17, wherein the polynucleotide has at least one nucleotide substitution of nucleotides corresponding to positions 334 to 339 in the nucleotide sequence of SEQ ID NO: 2.

19. The polynucleotide of claim 17, wherein the polynucleotide comprises a substitution of nucleotides corresponding to positions 334 to 336 in the nucleotide sequence of SEQ ID NO: 2 with TTG, TTA, CTT, CTC, or CTA; a substitution of nucleotides corresponding to positions 337 to 339 in the nucleotide sequence of SEQ ID NO: 2 with TTG, TTA, CTT, CTC, or CTA; or a combination thereof.

20. The polynucleotide of claim 17, wherein the polynucleotide encoding cellulose synthase has a synonymous nucleic acid alteration of a codon encoding an amino acid residue corresponding to position L112, L113, L114, A115, E116, or L117, or a combination thereof, in an amino acid sequence of SEQ ID NO: 1.
